(19) United States
(12) Patent Application Publication
(10) Pub. No.: US 2005/0037070 A1
(43) Pub. Date: Feb. 17, 2005

(54) Pharmaceutical formulatins useful for inhibiting acid secretion and methods for making and using them

(75) Inventors: Warren Hall, San Diego, CA (US); Kay Olmstead, San Diego, CA (US); Laura Weston, San Diego, CA (US)

Correspondence Address: WILSON SONSINI GOODRICH & ROSATI, 650 PAGE MILL ROAD, PALO ALTO, CA, 943041050, US

(73) Assignee: Santarus, Inc.

(21) Appl. No.: 10/893,203

(22) Filed: Jul. 16, 2004

Related U.S. Application Data
(60) Provisional application No. 60/488,321, filed on Jul. 18, 2003.

(51) Int. Cl. A61K 9/20; A61K 9/46; A61K 33/06

(52) U.S. Cl. 424/464; 424/682; 514/338

(57) ABSTRACT

In one general aspect of the present invention, pharmaceutical formulations comprising both a proton pump inhibitor microencapsulated with a material that enhances the shelf-life of the pharmaceutical composition and one or more antacid are described. In another general aspect of the present invention, pharmaceutical formulations comprising both a proton pump inhibitor microencapsulated with a taste-masking material and one or more antacid are described.

This application claims benefit under 35 U.S.C. § 119(e) of U.S. Provisional Application No. 60/488,321, filed Jul. 18, 2003, the contents of which are fully incorporated by reference herewith.

### FIELD OF THE INVENTION

The present invention is related to pharmaceutical formulations comprising an antacid and a proton pump inhibitor microencapsulated with (1) a material that enhances the shelf-life of the composition, or (2) a taste-masking material. In addition, methods for manufacture of the pharmaceutical formulations; uses of the pharmaceutical formulations in treating disease; and combinations of the pharmaceutical formulations with other therapeutic agents are described.

### BACKGROUND OF THE INVENTION

Upon ingestion, most acid-labile pharmaceutical compounds must be protected from contact with acidic stomach secretions to maintain their pharmaceutical activity. To accomplish this, compositions with enteric-coatings have been designed to dissolve at a pH to ensure that the drug is released in the proximal region of the small intestine (duodenum), rather than the acidic environment of the stomach. However, due to the pH-dependent attributes of these enteric-coated compositions and the uncertainty of gastric retention time, in-vivo performance as well as both inter- and intra-subject variability are all major set backs of using enteric-coated systems for the controlled release of a drug.

In addition, Phillips et al. has described non-enteric coated pharmaceutical compositions. These compositions, which allow for the immediate release of the pharmaceutically active ingredient into the stomach, involve the administration of one or more buffering agents with an acid labile pharmaceutical agent, such as a proton pump inhibitor. The buffering agent is thought to prevent substantial degradation of the acid labile pharmaceutical agent in the acidic environment of the stomach by raising the pH. See, e.g., U.S. Pat. Nos. 5,840,737; 6,489,346; 6,645,988; and 6,699,885.

A class of acid-labile pharmaceutical compounds that are administered as enteric-coated dosage forms are proton pump inhibiting agents. Exemplary proton pump inhibitors include, omeprazole (Prilosec®), lansoprazole (Prevacid®), esomeprazole (Nexium®), rabeprazole (Aciphex®), pantoprazole (Protonix®), pariprazole, tentaprazole, and leminoprazole. The drugs of this class suppress gastrointestinal acid secretion by the specific inhibition of the H⁺/K⁺-ATPase enzyme system (proton pump) at the secretory surface of the gastrointestinal parietal cell. Most proton pump inhibitors are susceptible to acid degradation and, as such, are rapidly destroyed as pH falls to an acidic level. Therefore, if the enteric-coating of these formulated products is disrupted (e.g., trituration to compound a liquid, or chewing the capsule or tablet) or the buffering agent fails to sufficiently neutralize the gastrointestinal pH, the drug will be exposed to degradation by the gastrointestinal acid in the stomach.

Omeprazole is one example of a proton pump inhibitor which is a substituted bicyclic aryl-imidazole, 5-methoxy-2-[(4-methoxy-3,5-dimethyl-2-pyridinyl) methyl] sulfinyl]-1H-benzimidazole, that inhibits gastrointestinal acid secretion. U.S. Pat. No. 4,786,505 to Lovgren et al. teaches that a pharmaceutical oral solid dosage form of omeprazole must be protected from contact with acidic gastrointestinal juice by an enteric-coating to maintain its pharmaceutical activity and describes an enteric-coated omeprazole preparation containing one or more subcoats between the core material and the enteric-coating.

Proton pump inhibitors are typically prescribed for short-term treatment of active duodenal ulcers, gastrointestinal ulcers, gastro esophageal reflux disease (GERD), severe erosive esophagitis, poorly responsive symptomatic GERD, and pathological hypersecretory conditions such as Zollinger Ellison syndrome. These above-listed conditions commonly arise in healthy or critically ill patients of all ages, and may be accompanied by significant upper gastrointestinal bleeding.

It is believed that omeprazole, lansoprazole and other proton pump inhibiting agents reduce gastrointestinal acid production by inhibiting H⁺/K⁺-ATPase of the parietal cell the final common pathway for gastrointestinal acid secretion. See, e.g., Fellenius et al., Substituted Benzimidazoles Inhibit Gastrointestinal Acid Secretion by Blocking H⁺/K⁺-ATPase, Nature, 290: 159-161 (1981); Wallmark et al., The Relationship Between Gastrointestinal Acid Secretion and Gastrointestinal H⁺/K⁺-ATPase Activity, J. Biol. Chem., 260: 13681-13684 (1985); and Fryklund et al., Function and Structure of Parietal Cells After H⁺/K⁺-ATPase Blockade, Am. J. Physiol., 254 (1988).

Proton pump inhibitors have the ability to act as weak bases which reach parietal cells from the blood and diffuse into the secretory canaliculi. There the drugs become protonated and thereby trapped. The protonated compound can then rearrange to form a sulfenamide which can covalently interact with sulfhydryl groups at critical sites in the extra cellular (luminal) domain of the membrane-spanning H⁺/K⁺-ATPase. See, e.g., Hardman et al., Goodman & Gilman 's The Pharmacological Basis of Therapeutics, 907 (9th ed. 1996). As such, proton pump inhibitors are prodrugs that must be activated to be effective. The specificity of the effects of proton pump inhibiting agents is also dependent upon: (a) the selective distribution of H⁺/K⁺-ATPase; (b) the requirement for acidic conditions to catalyze generation of the reactive inhibitor; and (c) the trapping of the protonated drug and the cationic sulfenamide within the acidic canaliculi and adjacent to the target enzyme. See, e.g., Hardman et al.

Still, there remains a need for a pharmaceutical formulation that releases a proton pump inhibitor into the gastrointestinal tract for absorption of an intact, non-acid degraded or non-acid reacted form of a proton pump inhibitor into the bloodstream of a subject in either a fed or fasting state which exhibits enhanced shelf-life stability and improved patient compliance. The discussion that follows discloses pharmaceutical formulations comprising microencapsulated proton pump inhibitors and one or more antacids which help to fulfill these needs.

### BRIEF DESCRIPTION OF THE DRAWINGS

FIG. 1 is a graph comparing the pharmacokinetic release profiles of omeprazole of Prilosec, naked omeprazole and antacid tablet (31 mEq), omeprazole microencapsulated with Klucel and antacid tablet (31 mEq), and omeprazole microencapsulated with Methocel and antacid tablet (31 mEq) in human.

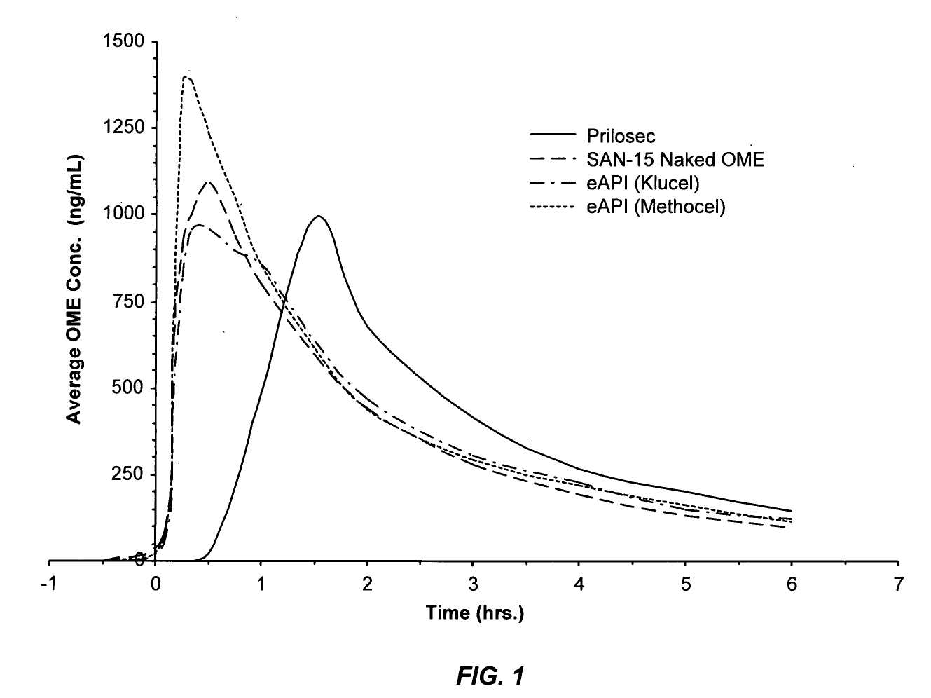

FIGS. 2A and 2B are SEM picture of microencapsulated omeprazole with Klucel.

### SUMMARY OF THE INVENTION

Provided herein are pharmaceutical formulations having enhanced shelf-lives comprising, at least one acid labile proton pump inhibitor which is microencapsulated with a material that enhances the shelf-life of the pharmaceutical formulation; and at least one antacid; wherein an initial serum concentration of the proton pump inhibitor is greater than about 0.1 μg/ml at any time within about 30 minutes after administration of the pharmaceutical formulation. Also provided herein are taste-masked pharmaceutical formulations comprising at least one acid labile proton pump inhibitor which is microencapsulated with a taste-masking material; and at least one antacid; wherein an initial serum concentration of the proton pump inhibitor is greater than about 0.1 μg/ml at any time within about 30 minutes after administration of the pharmaceutical formulation.

In various embodiments provided herein, the proton pump inhibitor is microencapsulated with one or more compounds selected from cellulose hydroxypropyl ethers; low-substituted hydroxypropyl ethers; cellulose hydroxypropyl methyl ethers; methylcellulose polymers; ethylcelluloses and mixtures thereof; polyvinyl alcohol; hydroxyethylcelluloses; carboxymethylcelluloses and salts of carboxymethylcelluloses; polyvinyl alcohol and polyethylene glycol co-polymers; monoglycerides; triglycerides; polyethylene glycols, modified food starch, acrylic polymers; mixtures of acrylic polymers with cellulose ethers; cellulose acetate phthalate; sepifilms, cyclodextrins; and mixtures thereof.

In various embodiments provided herein, the proton pump inhibitor is microencapsulated with one or more additives to enhance the processing or performance of microencapsulation. Such additives maybe pH modifier, plastersizer, antioxidant, or sweetener or flavor.

In other embodiments, the at least one antacid comprises at least one soluble antacid. In some embodiments, the soluble antacid is sodium bicarbonate. In various embodiments, the at least one buffer is selected from sodium bicarbonate, calcium carbonate, sodium carbonate, magnesium oxide, magnesium hydroxide, magnesium carbonate, aluminum hydroxide, and mixtures thereof.

Provided herein are methods of extending the shelf-life of pharmaceutical formulations comprising microencapsulating at least one acid labile proton pump inhibitor with a material that enhances the shelf-life; and combining the microencapsulated acid labile proton pump inhibitor with at least one antacid. Also provided herein are methods of masking the taste of a pharmaceutical formulation comprising microencapsulating at least one acid labile proton pump inhibitor with a taste-masking material; and combining the microencapsulated acid labile proton pump inhibitor with an antacid.

In various embodiments of the present invention, the pharmaceutical formulations may further comprise one or more excipients selected from parietal cell activators, organic solvents, erosion facilitators, diffusion facilitators, antioxidants, flavoring agents and carrier materials selected from binders, suspending agents, disintegration agents, filling agents, surfactants, solubilizers, stabilizers, lubricants, wetting agents, diluents, anti-adherents, and antifoaming agents.

### DETAILED DESCRIPTION OF THE INVENTION

The present invention is directed to pharmaceutical formulations exhibiting enhanced shelf-life stability and/or improved taste masking properties useful for the treatment of a disease, condition or disorder. Methods of treatment using the pharmaceutical formulations of the present invention are also described.

It has been discovered that pharmaceutical compositions comprising (1) an acid labile proton pump inhibitor which is microencapsulated with a material that enhances the shelf-life of the pharmaceutical composition together with (2) one or more antacid, provide superior performance by enhancing shelf-life stability of the pharmaceutical formulation during manufacturing and storage.

Certain taste-masking materials have also been discovered which, when used in the pharmaceutical formulations provide (1) more palatable forms of the drug by blocking the contact of the unpleasant taste of the pharmaceutical agent from the contact of the taste receptor, thereby increasing patient compliance; and/or (2) require lower amounts of traditional flavoring agents.

To more readily facilitate an understanding of the invention and its preferred embodiments, the meanings of terms used herein will become apparent from the context of this specification in view of common usage of various terms and the explicit definitions of other terms provided in the glossary below or in the ensuing description.

Glossary

As used herein, the terms “comprising,” “including,” and “such as” are used in their open, non-limiting sense.

The term “about” is used synonymously with the term “approximately.” Illustratively, the use of the term “about” indicates that values slightly outside the cited values, i.e., plus or minus 0.1% to 10%, which are also effective and safe. Such dosages are thus encompassed by the scope of the claims reciting the terms “about” and “approximately.”

The phrase “acid-labile pharmaceutical agent” refers to any pharmacologically active drug subject to acid catalyzed degradation.

“Aftertaste” is a measurement of all sensation remaining after swallowing. Aftertaste can be measured, e.g., from 30 seconds after swallowing, 1 minutes after swallowing, 2 minutes after swallowing, 3 minutes after swallowing, 4 minutes after swallowing, 5 minutes after swallowing, and the like.

“Amplitude” is the initial overall perception of the flavors balance and fullness. The amplitude scale is 0-none, 1-low, 2-moderate, and 3-high.

“Anti-adherents,” “glidants,” or “anti-adhesion” agents prevent components of the formulation from aggregating or sticking and improve flow characteristics of a material. Such compounds include, e.g., colloidal silicon dioxide such as Cab-o-sil®; tribasic calcium phosphate, talc, corn starch, DL-leucine, sodium lauryl sulfate, magnesium stearate, calcium stearate, sodium stearate, kaolin, and micronized amorphous silicon dioxide (Syloid®) and the like.

“Antifoaming agents” reduce foaming during processing which can result in coagulation of aqueous dispersions, bubbles in the finished film, or generally impair processing. Exemplary anti-foaming agents include silicon emulsions or sorbitan sesquoleate.

“Antioxidants” include, e.g., butylated hydroxytoluene (BHT), sodium ascorbate, and tocopherol.

“Binders” impart cohesive qualities and include, e.g., alginic acid and salts thereof; cellulose derivatives such as carboxymethylcellulose, methylcellulose (e.g., Methocel®), hydroxypropylmethylcellulose, hydroxyethylcellulose, hydroxypropylcellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®, glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and the like.

“Bioavailability” refers to the extent to which an active moiety, e.g., drug, prodrug, or metabolite, is absorbed into the general circulation and becomes available at the site of drug action in the body. Thus, a proton pump inhibitor administered through IV is 100% bioavailable. “Oral bioavailability” refers to the extent to with the proton pump inhibitor is absorbed into the general circulation and becomes available at the site of the drug action in the body when the pharmaceutical formulation is taken orally.

“Bioequivalence” or “bioequivalent” means that the area under the serum concentration time curve (AUC) and the peak serum concentration (Cₘₐₓ) are each within 80% and 120%.

“Carrier materials” include any commonly used excipients in pharmaceutics and should be selected on the basis of compatibility with the proton pump inhibitor and the release profile properties of the desired dosage form. Exemplary carrier materials include, e.g., binders, suspending agents, disintegration agents, filling agents, surfactants, solubilizers, stabilizers, lubricants, wetting agents, diluents, and the like. “Pharmaceutically compatible carrier materials” may comprise, e.g., acacia, gelatin, colloidal silicon dioxide, calcium glycerophosphate, calcium lactate, maltodextrin, glycerine, magnesium silicate, sodium caseinate, soy lecithin, sodium chloride, tricalcium phosphate, dipotassium phosphate, sodium stearoyl lactylate, carrageenan, monoglyceride, diglyceride, pregelatinized starch, and the like. See, e.g., Remington: The Science and Practice of Pharmacy, Nineteenth Ed (Easton, Pa.: Mack Publishing Company, 1995); Hoover, John E., Remington 's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

“Character notes” include, e.g., aromatics, basis tastes, and feeling factors. The intensity of the character note can be scaled from 0-none, 1-slight, 2-moderate, or 3-strong.

A “derivative” is a compound that is produced from another compound of similar structure by the replacement of substitution of an atom, molecule or group by another suitable atom, molecule or group. For example, one or more hydrogen atom of a compound may be substituted by one or more alkyl, acyl, amino, hydroxyl, halo, haloalkyl, aryl, heteroaryl, cycloaolkyl, heterocycloalkyl, or heteroalkyl group to produce a derivative of that compound.

“Diffusion facilitators” and “dispersing agents” include materials that control the diffusion of an aqueous fluid through a coating. Exemplary diffusion facilitators/dispersing agents include, e.g., hydrophilic polymers, electrolytes, Tween® 60 or 80, PEG and the like. Combinations of one or more erosion facilitator with one or more diffusion facilitator can also be used in the present invention.

“Diluents” increase bulk of the composition to facilitate compression. Such compounds include e.g., lactose; starch; mannitol; sorbitol; dextrose; microcrystalline cellulose such as Avicel®; dibasic calcium phosphate; dicalcium phosphate dihydrate; tricalcium phosphate; calcium phosphate; anhydrous lactose; spray-dried lactose; pregelatinzed starch; compressible sugar, such as Di-Pac® (Amstar); mannitol; hydroxypropylmethylcellulose; sucrose-based diluents; confectioner's sugar; monobasic calcium sulfate monohydrate; calcium sulfate dihydrate; calcium lactate trihydrate; dextrates; hydrolyzed cereal solids; amylose; powdered cellulose; calcium carbonate; glycine; kaolin; mannitol; sodium chloride; inositol; bentonite; and the like.

The term “disintegrate” includes both the dissolution and dispersion of the dosage form when contacted with gastrointestinal fluid.

“Disintegration agents” facilitate the breakup or disintegration of a substance. Examples of disintegration agents include a starch, e.g., a natural starch such as corn starch or potato starch, a pregelatinized starch such as National 1551 or Amijel®, or sodium starch glycolate such as Promogel® or Explotab®; a cellulose such as a wood product, methylcrystalline cellulose, e.g., Avicel®, Avicel® PHI 01, Avicel® PH102, Avicel® PH105, Elcema® P100, Emcocel®, Vivacel®, Ming Tia®, and Solka-Floc®, methylcellulose, croscarmellose, or a cross-linked cellulose, such as cross-linked sodium carboxymethylcellulose (Ac-Di-Sol®), cross-linked carboxymethylcellulose, or cross-linked croscarmellose; a cross-linked starch such as sodium starch glycolate; a cross-linked polymer such as crospovidone; a cross-linked polyvinylpyrrolidone; alginate such as alginic acid or a salt of alginic acid such as sodium alginate; a clay such as Veegum® HV (magnesium aluminum silicate); a gum such as agar, guar, locust bean, Karaya, pectin, or tragacanth; sodium starch glycolate; bentonite; a natural sponge; a surfactant; a resin such as a cation-exchange resin; citrus pulp; sodium lauryl sulfate; sodium lauryl sulfate in combination starch; and the like.

“Drug absorption” or “absorption” refers to the process of movement from the site of administration of a drug toward the systemic circulation, e.g., into the bloodstream of a subject.

An “enteric coating” is a substance that remains substantially intact in the stomach but dissolves and releases the drug once the small intestine is reached. Generally, the enteric coating comprises a polymeric material that prevents release in the low pH environment of the stomach but that ionizes at a slightly higher pH, typically a pH of 4 or 5, and thus dissolves sufficiently in the small intestines to gradually release the active agent therein.

The “enteric form of the proton pump inhibitor” is intended to mean that some or most of the proton pump inhibitor has been enterically coated to ensure that at least some of the drug is released in the proximal region of the small intestine (duodenum), rather than the acidic environment of the stomach.

“Erosion facilitators” include materials that control the erosion of a particular material in gastrointestinal fluid. Erosion facilitators are generally known to those of ordinary skill in the art. Exemplary erosion facilitators include, e.g., hydrophilic polymers, electrolytes, proteins, peptides, and amino acids.

“Filling agents” include compounds such as lactose, calcium carbonate, calcium phosphate, dibasic calcium phosphate, calcium sulfate, microcrystalline cellulose, cellulose powder, dextrose, dextrates, dextran, starches, pregelatinized starch, sucrose, xylitol, lactitol, mannitol, sorbitol, sodium chloride, polyethylene glycol, and the like.

“Flavoring agents” or “sweeteners” useful in the pharmaceutical compositions of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrthetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, Swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof.

“Gastrointestinal fluid” is the fluid of stomach secretions of a subject or the saliva of a subject after oral administration of a composition of the present invention, or the equivalent thereof. An “equivalent of stomach secretion” includes, e.g., an in vitro fluid having similar content and/or pH as stomach secretions such as a 1% sodium dodecyl sulfate solution or 0.1N HCl solution in water.

“Half-life” refers to the time required for the plasma drug concentration or the amount in the body to decrease by 50% from its maximum concentration.

“Lubricants” are compounds that prevent, reduce or inhibit adhesion or friction of materials. Exemplary lubricants include, e.g., stearic acid; calcium hydroxide; talc; sodium stearyl fumerate; a hydrocarbon such as mineral oil, or hydrogenated vegetable oil such as hydrogenated soybean oil (Sterotex®); higher fatty acids and their alkali-metal and alkaline earth metal salts, such as aluminum, calcium, magnesium, zinc, stearic acid, sodium stearates, glycerol, talc, waxes, Stearowet®, boric acid, sodium benzoate, sodium acetate, sodium chloride, leucine, a polyethylene glycol or a methoxypolyethylene glycol such as Carbowax™, sodium oleate, glyceryl behenate, polyethylene glycol, magnesium or sodium lauryl sulfate, colloidal silica such as Syloid™, Carb-O-Sil®, a starch such as corn starch, silicone oil, a surfactant, and the like.

A “measurable serum concentration” or “measurable plasma concentration” describes the blood serum or blood plasma concentration, typically measured in mg, μg, or ng of therapeutic agent per ml, dl, or 1 of blood serum, of a therapeutic agent that is absorbed into the bloodstream after administration. One of ordinary skill in the art would be able to measure the serum concentration or plasma concentration of a proton pump inhibitor or a prokinetic agent. See, e.g., Gonzalez H. et al., J. Chromatogr. B. Analyt. Technol. Biomed. Life Sci., vol. 780, pp 459-65, (Nov. 25, 2002).

“Parietal cell activators” or “activators” stimulate the parietal cells and enhance the pharmaceutical activity of the proton pump inhibitor. Parietal cell activators include, e.g., chocolate; alkaline substances such as sodium bicarbonate; calcium such as calcium carbonate, calcium gluconate, calcium hydroxide, calcium acetate and calcium glycerophosphate; peppermint oil; spearmint oil; coffee; tea and colas (even if decaffeinated); caffeine; theophylline; theobromine; amino acids (particularly aromatic amino acids such as phenylalanine and tryptophan); and combinations thereof.

“Pharmacodynamics” refers to the factors that determine the biologic response observed relative to the concentration of drug at a site of action.

“Pharmacokinetics” refers to the factors that determine the attainment and maintenance of the appropriate concentration of drug at a site of action.

“Plasma concentration” refers to the concentration of a substance in blood plasma or blood serum of a subject. It is understood that the plasma concentration of a therapeutic agent may vary many-fold between subjects, due to variability with respect to metabolism of therapeutic agents. In accordance with one aspect of the present invention, the plasma concentration of a proton pump inhibitors and/or prokinetic agent may vary from subject to subject. Likewise, values such as maximum plasma concentration (Cₘₐₓ) or time to reach maximum serum concentration (Tₘₐₓ), or area under the serum concentration time curve (AUC) may vary from subject to subject. Due to this variability, the amount necessary to constitute “a therapeutically effective amount” of proton pump inhibitor, prokinetic agent, or other therapeutic agent, may vary from subject to subject. It is understood that when mean plasma concentrations are disclosed for a population of subjects, these mean values may include substantial variation.

“Plasticizers” are compounds used to soften the microencapsulation material or film coatings to make them less brittle. Suitable plasticizers include, e.g., polyethylene glycols such as PEG 300, PEG 400, PEG 600, PEG 1450, PEG 3350, and PEG 800, stearic acid, propylene glycol, oleic acid, and triacetin.

“Prevent” or “prevention” when used in the context of a gastric acid related disorder means no gastrointestinal disorder or disease development if none had occurred, or no further gastrointestinal disorder or disease development if there had already been development of the gastrointestinal disorder or disease. Also considered is the ability of one to prevent some or all of the symptoms associated with the gastrointestinal disorder or disease.

A “prodrug” refers to a drug or compound in which the pharmacological action results from conversion by metabolic processes within the body. Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs may be designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987.

“Proton pump inhibitor product” refers to a product sold on the market. Proton pump inhibitor products include, for example, Priolosec®, Nexium®, Prevacid®, Protonic®, and Aciphex®.

“Serum concentration” refers to the concentration of a substance such as a therapeutic agent, in blood plasma or blood serum of a subject. It is understood that the serum concentration of a therapeutic agent may vary many-fold between subjects, due to variability with respect to metabolism of therapeutic agents. In accordance with one aspect of the present invention, the serum concentration of a proton pump inhibitors and/or prokinetic agent may vary from subject to subject. Likewise, values such as maximum serum concentration (Cₘₐₓ) or time to reach maximum serum concentration (Tₘₐₓ), or total area under the serum concentration time curve (AUC) may vary from subject to subject. Due to this variability, the amount necessary to constitute “a therapeutically effective amount” of proton pump inhibitor, prokinetic agent, or other therapeutic agent, may vary from subject to subject. It is understood that when mean serum concentrations are disclosed for a population of subjects, these mean values may include substantial variation.

“Solubilizers” include compounds such as citric acid, succinic acid, fumaric acid, malic acid, tartaric acid, maleic acid, glutaric acid, sodium bicarbonate, sodium carbonate and the like.

“Stabilizers” include compounds such as any antioxidation agents, buffers, acids, and the like.

“Suspending agents” or “thickening agents” include compounds such as polyvinylpyrrolidone, e.g., polyvinylpyrrolidone K12, polyvinylpyrrolidone K17, polyvinylpyrrolidone K25, or polyvinylpyrrolidone K30; polyethylene glycol, e.g., the polyethylene glycol can have a molecular weight of about 300 to about 6000, or about 3350 to about 4000, or about 7000 to about 5400; sodium carboxymethylcellulose; methylcellulose; hydroxy-propylmethylcellulose; polysorbate-80; hydroxyethylcellulose; sodium alginate; gums, such as, e.g., gum tragacanth and gum acacia; guar gum; xanthans, including xanthan gum; sugars; cellulosics, such as, e.g., sodium carboxymethylcellulose, methylcellulose, sodium carboxymethylcellulose, hydroxypropylmethylcellulose, hydroxyethylcellulose; polysorbate-80; sodium alginate; polyethoxylated sorbitan monolaurate; polyethoxylated sorbitan monolaurate; povidone and the like.

“Surfactants” include compounds such as sodium lauryl sulfate, sorbitan monooleate, polyoxyethylene sorbitan monooleate, polysorbates, polaxomers, bile salts, glyceryl monostearate, copolymers of ethylene oxide and propylene oxide, e.g., Pluronic® (BASF); and the like.

A “therapeutically effective amount” or “effective amount” is that amount of a pharmaceutical agent to achieve a pharmacological effect. The term “therapeutically effective amount” includes, for example, a prophylactically effective amount. An “effective amount” of a proton pump inhibitor is an amount effective to achieve a desired pharmacologic effect or therapeutic improvement without undue adverse side effects. For example, an effective amount of a proton pump inhibitor refers to an amount of proton pump inhibitor that reduces acid secretion, or raises gastrointestinal fluid pH, or reduces gastrointestinal bleeding, or reduces the need for blood transfusion, or improves survival rate, or provides for a more rapid recovery from a gastric acid related disorder. The effective amount of a pharmaceutical agent will be selected by those skilled in the art depending on the particular patient and the disease level. It is understood that “an effect amount” or “a therapeutically effective amount” can vary from subject to subject, due to variation in metabolism of therapeutic agents such as proton pump inhibitors and/or prokinetic agents, age, weight, general condition of the subject, the condition being treated, the severity of the condition being treated, and the judgment of the prescribing physician.

“Total intensity of aroma” is the overall immediate impression of the strength of the aroma and includes both aromatics and nose feel sensations.

“Total intensity of flavor” is the overall immediate impression of the strength of the flavor including aromatics, basic tastes and mouth feel sensations.

“Treat” or “treatment” as used in the context of a gastric acid related disorder refers to any treatment of a disorder or disease associated with a gastrointestinal disorder, such as preventing the disorder or disease from occurring in a subject which may be predisposed to the disorder or disease, but has not yet been diagnosed as having the disorder or disease; inhibiting the disorder or disease, e.g., arresting the development of the disorder or disease, relieving the disorder or disease, causing regression of the disorder or disease, relieving a condition caused by the disease or disorder, or stopping the symptoms of the disease or disorder. Thus, as used herein, the term “treat” is used synonymously with the term “prevent.”

“Wetting agents” include compounds such as oleic acid, glyceryl monostearate, sorbitan monooleate, sorbitan monolaurate, triethanolamine oleate, polyoxyethylene sorbitan monooleate, polyoxyethylene sorbitan monolaurate, sodium oleate, sodium lauryl sulfate, and the like.

Proton Pump Inhibitors

The terms “proton pump inhibitor,” “PPI,” and “proton pump inhibiting agent” can be used interchangeably to describe any acid labile pharmaceutical agent possessing pharmacological activity as an inhibitor of H+/K+-ATPase. A proton pump inhibitor may, if desired, be in the form of free base, free acid, salt, ester, hydrate, anhydrate, amide, enantiomer, isomer, tautomer, prodrug, polymorph, derivative, or the like, provided that the free base, salt, ester, hydrate, amide, enantiomer, isomer, tautomer, prodrug, or any other pharmacologically suitable derivative is therapeutically active.

In various embodiments, the proton pump inhibitor can be a substituted bicyclic aryl-imidazole, wherein the aryl group can be, e.g., a pyridine, a phenyl, or a pyrimidine group and is attached to the 4- and 5-positions of the imidazole ring. Proton pump inhibitors comprising a substituted bicyclic aryl-imidazoles include, but are not limited to, omeprazole, hydroxyomeprazole, esomeprazole, lansoprazole, pantoprazole, rabeprazole, dontoprazole, habeprazole, perprazole, tenatoprazole, ransoprazole, pariprazole, leminoprazole, or a free base, free acid, salt, hydrate, ester, amide, enantiomer, isomer, tautomer, polymorph, prodrug, or derivative thereof. See, e.g., The Merck Index, Merck & Co. Rahway, N.J. (2001).

Other proton pump inhibitors include but are not limited to: soraprazan (Altana);
ilaprazole (U.S. Pat. No. 5,703,097) (Il-Yang); AZD-0865 (AstraZeneca); YH-1885 (PCT Publication WO 96/05177) (SB-641257) (2-pyrimidinamine, 4-(3,4-dihydro-1-methyl-2(1H)-isoquinolinyl)-N-(4-fluorophenyl)-5,6-dimethyl-monohydrochloride)(YuHan); BY-112 (Altana); SPI-447 (Imidazo(1,2-a)thieno(3,2-c)pyridin-3-amine,5-methyl-2-(2-methyl-3-thienyl) (Shinnippon); 3-hydroxymethyl-2methyl-9-phenyl-7H-8,9-dihydro-pyrano(2,3-c)-imidazo(1,2-a)pyridine (PCT Publication WO 95/27714) (AstraZeneca); Pharmaprojects No. 4950 (3-hydroxymethyl-2-methyl-9-phenyl-7H-8,9-dihydro-pyrano(2,3-c)-imidazo(1,2-a)pyridine) (AstraZeneca, ceased) WO 95/27714; Pharmaprojects No. 4891 (EP 700899) (Aventis); Pharmaprojects No. 4697 (PCT Publication WO 95/32959) (AstraZeneca); H-335/25 (AstraZeneca); T-330 (Saitama 335) (Pharmacological Research Lab); Pharmaprojects No. 3177 (Roche); BY-574 (Altana); Pharmaprojects No. 2870 (Pfizer); AU-1421 (EP 264883) (Merck); AU-2064 (Merck); AY-28200 (Wyeth); Pharmaprojects No. 2126 (Aventis); WY-26769 (Wyeth); pumaprazole (PCT Publication WO 96/05199) (Altana); YH-1238 (YuHan); Pharmaprojects No. 5648 (PCT Publication WO 97/32854) (Dainippon); BY-686 (Altana); YM-020 (Yamanouchi); GYKI-34655 (Ivax); FPL-65372 (Aventis); Pharmaprojects No. 3264 (EP 509974) (AstraZeneca); nepaprazole (To a Eiyo); HN-11203 (Nycomed Pharma); OPC-22575; pumilacidin A (BMS); saviprazole (EP 234485) (Aventis); SKand F-95601 (GSK, discontinued); Pharmaprojects No. 2522 (EP 204215) (Pfizer); S-3337 (Aventis); RS-13232A (Roche); AU-1363 (Merck); SKand F-96067 (EP 259174) (Altana); SUN 8176 (Daiichi Phama); Ro-18-5362 (Roche); ufiprazole (EP 74341) (AstraZeneca); and Bay-p-1455 (Bayer); or a free base, free acid, salt, hydrate, ester, amide, enantiomer, isomer, tautomer, polymorph, prodrug, or derivative of these compounds.

Still other proton pump inhibitors contemplated by the present invention include those described in the following U.S. Pat. Nos. 4,628,098; 4,689,333; 4,786,505; 4,853,230; 4,965,269; 5,021,433; 5,026,560; 5,045,321; 5,093,132; 5,430,042; 5,433,959; 5,576,025; 5,639,478; 5,703,110; 5,705,517; 5,708,017; 5,731,006; 5,824,339; 5,855,914; 5,879,708; 5,948,773; 6,017,560; 6,123,962; 6,187,340; 6,296,875; 6,319,904; 6,328,994; 4,255,431; 4,508,905; 4,636,499; 4,738,974; 5,690,960; 5,714,504; 5,753,265; 5,817,338; 6,093,734; 6,013,281; 6,136,344; 6,183,776; 6,328,994; 6,479,075; 6,559,167.

Other substituted bicyclic aryl-imidazole compounds as well as their salts, hydrates, esters, amides, enantiomers, isomers, tautomers, polymorphs, prodrugs, and derivatives may be prepared using standard procedures known to those skilled in the art of synthetic organic chemistry. See, e.g., March, Advanced Organic Chemistry: Reactions, Mechanisms and Structure, 4th Ed. (New York: Wiley-Interscience, 1992); Leonard et al., Advanced Practical Organic Chemistry (1992); Howarth et al., Core Organic Chemistry (1998); and Weisermel et al., Industrial Organic Chemistry (2002).

“Pharmaceutically acceptable salts,” or “salts,” include, e.g., the salt of a proton pump inhibitor prepared from formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, stearic, salicylic, p-hydroxybenzoic, phenylacetic, mandelic, embonic, methanesulfonic, ethanesulfonic, benzenesulfonic, pantothenic, toluenesulfonic, 2-hydroxyethanesulfonic, sulfanilic, cyclohexylaminosulfonic, algenic, β-hydroxybutyric, galactaric and galacturonic acids.

In one embodiment, acid addition salts are prepared from the free base using conventional methodology involving reaction of the free base with a suitable acid. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like.

In other embodiments, an acid addition salt is reconverted to the free base by treatment with a suitable base. In a further embodiment, the acid addition salts of the proton pump inhibitors are halide salts, which are prepared using hydrochloric or hydrobromic acids. In still other embodiments, the basic salts are alkali metal salts, e.g., sodium salt.

Salt forms of proton pump inhibiting agents include, but are not limited to: a sodium salt form such as esomeprazole sodium, omeprazole sodium, rabeprazole sodium, pantoprazole sodium; or a magnesium salt form such as esomeprazole magnesium or omeprazole magnesium, described in U.S. Pat. No. 5,900,424; a calcium salt form; or a potassium salt form such as the potassium salt of esomeprazole, described in U.S. Patent Application No. 02/0198239 and U.S. Pat. No. 6,511,996. Other salts of esomeprazole are described in U.S. Pat. Nos. 4,738,974 and 6,369,085. Salt forms of pantoprazole and lansoprazole are discussed in U.S. Pat. Nos. 4,758,579 and 4,628,098, respectively.

In one embodiment, preparation of esters involves functionalizing hydroxyl and/or carboxyl groups that may be present within the molecular structure of the drug. In one embodiment, the esters are acyl-substituted derivatives of free alcohol groups, e.g., moieties derived from carboxylic acids of the formula RCOOR₁ where R₁ is a lower alkyl group. Esters can be reconverted to the free acids, if desired, by using conventional procedures such as hydrogenolysis or hydrolysis.

“Amides” may be prepared using techniques known to those skilled in the art or described in the pertinent literature. For example, amides may be prepared from esters, using suitable amine reactants, or they may be prepared from an anhydride or an acid chloride by reaction with an amine group such as ammonia or a lower alkyl amine.

“Tautomers” of substituted bicyclic aryl-imidazoles include, e.g., tautomers of omeprazole such as those described in U.S. Pat. Nos. 6,262,085; 6,262,086; 6,268,385; 6,312,723; 6,316,020; 6,326,384; 6,369,087; and 6,444,689; and U.S. Patent Publication No. 02/0156103.

An exemplary “isomer” of a substituted bicyclic aryl-imidazole is the isomer of omeprazole including but not limited to isomers described in: Oishi et al., Acta Cryst. (1989), C45, 1921-1923; U.S. Pat. No. 6,150,380; U.S. Patent Publication No. 02/0156284; and PCT Publication No. WO 02/085889.

Exemplary “polymorphs” include, but are not limited to, those described in PCT Publication No. WO 92/08716, and U.S. Pat. Nos. 4,045,563; 4,182,766; 4,508,905; 4,628,098; 4,636,499; 4,689,333; 4,758,579; 4,783,974; 4,786,505; 4,808,596; 4,853,230; 5,026,560; 5,013,743; 5,035,899; 5,045,321; 5,045,552; 5,093,132; 5,093,342; 5,433,959; 5,464,632; 5,536,735; 5,576,025; 5,599,794; 5,629,305; 5,639,478; 5,690,960; 5,703,110; 5,705,517; 5,714,504; 5,731,006; 5,879,708; 5,900,424; 5,948,773; 5,997,903; 6,017,560; 6,123,962; 6,147,103; 6,150,380; 6,166,213; 6,191,148; 5,187,340; 6,268,385; 6,262,086; 6,262,085; 6,296,875; 6,316,020; 6,328,994; 6,326,384; 6,369,085; 6,369,087; 6,380,234; 6,428,810; 6,444,689; and 6,462,0577.

Micronized Proton Pump Inhibitor

Particle size of the proton pump inhibitor can affect the solid dosage form in numerous ways. Because decreased particle size increases in surface area (S), the particle size reduction provides an increase in the rate of dissolution (dM/dt) as expressed in the Noyes-Whitney equation below:
dM/dt=dS/h(Cs−C)

M=mass of drug dissolved; t=time; D=diffusion coefficient of drug; S=effective surface area of drug particles; H=stationary layer thickness; Cs=concentration of solution at saturation; and C=concentration of solution at time t.

Because omeprazole, as well as other proton pump inhibitors, has poor water solubility, to aid the rapid absorption of the drug product, various embodiments of the present invention use micronized proton pump inhibitor in the microencapsulation.

In some embodiments, the average particle size of at least about 90% the micronized proton pump inhibitor is less than about 200 μm, 150 μm, 100 μm, 80 μm, 60 μm, 40 μm, or less than about 35 μm, or less than about 30 μm, or less than about 25 μm, or less than about 20 μm, or less than about 15 μm, or less than about 10 μm. In other embodiments, at least 80% of the micronized proton pump inhibitor has an average particle size of less than about 200 μm, 150 μm, 100 μm, 80 μm, 60 μm, 40 μm, or less than about 35 μm, or less than about 30 μm, or less than about 25 μm, or less than about 20 μm, or less than about 15 μm, or less than about 10 μm. In still other embodiments, at least 70% of the micronized proton pump inhibitor has an average particle size of less than about 200 μm, 150 μm, 100 μm, 80 μm, 60 μm, 40 μm, or less than about 35 μm, or less than about 30 μm, or less than about 25 μm, or less than about 20 μm, or less than about 15 μm, or less than about 10 μm.

Compositions are provided wherein the micronized proton pump inhibitor is of a size which allows greater than 75% of the proton pump inhibitor to be released within about 1 hour, or within about 50 minutes, or within about 40 minutes, or within about 30 minutes, or within about 20 minutes, or within about 10 minutes, or within about 5 minutes of dissolution testing. In another embodiment of the invention, the micronized proton pump inhibitor is of a size which allows greater than 90% of the proton pump inhibitor to be released within about 1 hour, or within about 50 minutes, or within about 40 minutes, or within about 30 minutes, or within about 20 minutes, or within about 10 minutes, or within about 5 minutes of dissolution testing. See U.S. Provisional Application No. 60/488,324 filed July 18, 2003, and any subsequent application claiming priority to this application, all of which are incorporated by reference in their entirety.

Antacids

The pharmaceutical composition of the invention comprises one or more antacids. A class of antacids useful in the present invention include, e.g., antacids possessing pharmacological activity as a weak base or a strong base. In one embodiment, the antacid, when formulated or delivered (e.g., before, during and/or after) with an proton pump inhibiting agent, functions to substantially prevent or inhibit the acid degradation of the proton pump inhibitor by gastrointestinal fluid for a period of time, e.g., for a period of time sufficient to preserve the bioavailability of the proton pump inhibitor administered. In one aspect of the present invention, the antacid includes a salt of a Group IA metal, including, e.g., a bicarbonate salt of a Group IA metal, a carbonate salt of a Group IA metal, an alkali earth metal antacid, an aluminum antacid, a calcium antacid, or a magnesium antacid.

Other antacids suitable for the present invention include, e.g., alkali (sodium and potassium) or alkali earth (calcium and magnesium) carbonates, phosphates, bicarbonates, citrates, borates, acetates, phthalates, tartrate, succinates and the like, such as sodium or potassium phosphate, citrate, borate, acetate, bicarbonate and carbonate.

In various embodiments, an antacid includes, e.g., an amino acid, an alkali salt of an amino acid, aluminum hydroxide, aluminum hydroxide/magnesium carbonate/calcium carbonate co-precipitate, aluminum magnesium hydroxide, aluminum hydroxide/magnesium hydroxide co-precipitate, aluminum hydroxide/sodium bicarbonate co-precipitate, aluminum glycinate, calcium acetate, calcium bicarbonate, calcium borate, calcium carbonate, calcium citrate, calcium gluconate, calcium glycerophosphate, calcium hydroxide, calcium lactate, calcium phthalate, calcium phosphate, calcium succinate, calcium tartrate, dibasic sodium phosphate, dipotassium hydrogen phosphate, dipotassium phosphate, disodium hydrogen phosphate, disodium succinate, dry aluminum hydroxide gel, L-arginine, magnesium acetate, magnesium aluminate, magnesium borate, magnesium bicarbonate, magnesium carbonate, magnesium citrate, magnesium gluconate, magnesium hydroxide, magnesium lactate, magnesium metasilicate aluminate, magnesium oxide, magnesium phthalate, magnesium phosphate, magnesium silicate, magnesium succinate, magnesium tartrate, potassium acetate, potassium carbonate, potassium bicarbonate, potassium borate, potassium citrate, potassium metaphosphate, potassium phthalate, potassium phosphate, potassium polyphosphate, potassium pyrophosphate, potassium succinate, potassium tartrate, sodium acetate, sodium bicarbonate, sodium borate, sodium carbonate, sodium citrate, sodium gluconate, sodium hydrogen phosphate, sodium hydroxide, sodium lactate, sodium phthalate, sodium phosphate, sodium polyphosphate, sodium pyrophosphate, sodium sesquicarbonate, sodium succinate, sodium tartrate, sodium tripolyphosphate, synthetic hydrotalcite, tetrapotassium pyrophosphate, tetrasodium pyrophosphate, tripotassium phosphate, trisodium phosphate, and trometarnol. (Based in part upon the list provided in The Merck Index, Merck & Co. Rahway, N.J. (2001)). In addition, due to the ability of proteins or protein hydrolysates to react with stomach acids, they too can serve as antacids in the present invention. Furthermore, combinations of the above mentioned antacids can be used in the pharmaceutical formulations described herein.

The antacids useful in the present invention also include antacids or combinations of antacids that interact with HCl (or other acids in the environment of interest) faster than the proton pump inhibitor interacts with the same acids. When placed in a liquid phase, such as water, these antacids produce and maintain a pH greater than the pKa of the proton pump inhibitor.

In various embodiments, the antacid is selected from sodium bicarbonate, sodium carbonate, calcium carbonate, magnesium oxide, magnesium hydroxide, magnesium carbonate, aluminum hydroxide, and mixtures thereof. In another embodiment, the antacid is sodium bicarbonate and is present in about 0.1 mEq/mg proton pump inhibitor to about 5 mEq/mg proton pump inhibitor. In yet another embodiment, the antacid is a mixture of sodium bicarbonate and magnesium hydroxide, wherein the sodium bicarbonate and magnesium hydroxide are each present in about 0.1 mEq/mg proton pump inhibitor to about 5 mEq/mg proton pump inhibitor. In still another embodiment, the antacid is a mixture of sodium bicarbonate, calcium carbonate, and magnesium hydroxide, wherein the sodium bicarbonate, calcium carbonate, and magnesium hydroxide are each present in about 0.1 mEq/mg proton pump inhibitor to about 5 mEq/mg of the proton pump inhibitor.

In various other embodiments of the present invention, the antacid is present in an amount of about 0.1 mEq/mg to about 5 mEq/mg of the proton pump inhibitor, or about 0.5 mEq/mg to about 3 mEq/mg of the proton pump inhibitor, or about 0.6 mEq/mg to about 2.5 mEq/mg of the proton pump inhibitor, or about 0.7 mEq/mg to about 2.0 mEq/mg of the proton pump inhibitor, or about 0.8 mEq/mg to about 1.8 mEq/mg of the proton pump inhibitor, or about 1.0 mEq/mg to about 1.5 mEq/mg of the proton pump inhibitor, or at least 0.5 mEq/mg of the proton pump inhibitor.

In another embodiment, the antacid is present in the pharmaceutical formulations of the present invention in an amount of about 0.1 mEq to about 15 mEq/mg of proton pump inhibitor, or about 0.1 mEq/mg of proton pump inhibitor, or about 0.5 mEq/mg of proton pump inhibitor, or about 1 mEq/mg of proton pump inhibitor, or about 2 mEq/mg of proton pump inhibitor, or about 2.5 mEq/mg of proton pump inhibitor, or about 3 mEq/mg of proton pump inhibitor, or about 3.5 mEq/mg of proton pump inhibitor, or about 4 mEq/mg of proton pump inhibitor, or about 4.5 mEq/mg of proton pump inhibitor, or about 5 mEq/mg of proton pump inhibitor, or about 6 mEq/mg of proton pump inhibitor, or about 7 mEq/mg of proton pump inhibitor, or about 8 mEq/mg of proton pump inhibitor, or about 9 mEq/mg of proton pump inhibitor, or about 10 mEq/mg of proton pump inhibitor, or about 11 mEq/mg of proton pump inhibitor, or about 12 mEq/mg of proton pump inhibitor, or about 13 mEq/mg of proton pump inhibitor, or about 14 mEq/mg of proton pump inhibitor, or about 15 mEq/mg of proton pump inhibitor.

In one embodiment, the antacid is present in the pharmaceutical formulations of the present invention in an amount of about 1 mEq to about 160 mEq per dose, or about 1 mEq, or about 5 mEq, or about 10 mEq, or about 15 mEq, or about 20 mEq, or about 25 mEq, or about 30 mEq, or about 35 mEq, or about 40 mEq, or about 45 mEq, or about 50 mEq, or about 60 mEq, or about 70 mEq, or about 80 mEq, or about 90 mEq, or about 100 mEq, or about 110 mEq, or about 120 mEq, or about 130 mEq, or about 140 mEq, or about 150 mEq, or about 160 mEq per dose.

In another embodiment, the antacid is present in an amount of more than about 5 times, or more than about 10 times, or more than about 20 times, or more than about 30 times, or more than about 40 times, or more than about 50 times, or more than about 60 times, or more than about 70 times, or more than about 80 times, or more than about 90 times, or more than about 100 times the amount of the proton pump inhibiting agent on a weight to weight basis in the composition.

In another embodiment, the amount of antacid present in the pharmaceutical formulation is between 200 and 3500 mg. In other embodiments, the amount of antacid present in the pharmaceutical formulation is about 200 mgs, or about 300 mgs, or about 400 mgs, or about 500 mgs, or about 600 mgs, or about 700 mgs, or about 800 mgs, or about 900 mgs, or about 1000 mgs, or about 1100 mgs, or about 1200 mgs, or about 1300 mgs, or about 1400 mgs, or about 1500 mgs, or about 1600 mgs, or about 1700 mgs, or about 1800 mgs, or about 1900 mgs, or about 2000 mgs, or about 2100 mgs, or about 2200 mgs, or about 2300 mgs, or about 2400 mgs, or about 2500 mgs, or about 2600 mgs, or about 2700 mgs, or about 2800 mgs, or about 2900 mgs, or about 3000 mgs, or about 3200 mgs, or about 3500 mgs.

In some embodiments, if the at least one buffering agent is a combination of two or more buffering agents, the combination comprises at least two non-amino acid buffering agents, wherein the combination of at least two non-amino acid buffering agents comprises substantially no aluminum hydroxide-sodium bicarbonate co-precipitate. In other embodiments, if the pharmaceutical composition comprises an amino acid buffering agent, the total amount of buffering agent present in the pharmaceutical composition is less than about 5 mEq, or less than about 4 mEq, or less than about 3 mEq. The phrase “amino acid buffering agent” as used herein includes amino acids, amino acid salts, and amino acid alkali salts including: glycine, alanine, threonine, isoleucine, valine, phenylalanine, glutamic acid, asparagininic acid, lysine, aluminum glycinate and/or lysine glutamic acid salt, glycine hydrochloride, L-alanine, DL-alanine, L-threonine, DL-threonine, L-isoleucine, L-valine, L-phenylalanine, L-glutamic acid, L-glutamic acid hydrochloride, L-glutamic acid sodium salt, L-asparaginic acid, L-asparaginic acid sodium salt, L-lysine and L-lysine-L-glutamic acid salt. The term “non-amino acid buffering agent” herein includes buffering agents as defined hereinabove but does not include amino acid buffering agents.

In other embodiments, the pharmaceutical composition comprises substantially no or no poly[phosphoryl/sulfon]-ated carbohydrate and is in the form of a solid dosage unit. In still another related embodiment, if such a composition comprises a poly[phosphoryl/sulfon]-ated carbohydrate (e.g. sucralfate or sucrose octasulfate), the weight ratio of poly[phosphoryl/sulfon]-ated carbohydrate to buffering agent is less than 1:5 (0.2), less than 1:10 (0.1) or less than 1:20 (0.05). Alternatively, the poly[phosphoryl/sulfon]-ated carbohydrate is present in the composition, if at all, in an amount less than 50 mg, less than 25 mg, less than 10 mg or less than 5 mg.

Also provided herein are pharmaceutical formulations comprising at least one soluble antacid. For example, in one embodiment, the antacid is sodium bicarbonate and is present in about 0.1 mEq/mg proton pump inhibitor to about 5 mEq/mg proton pump inhibitor. In another embodiment, the antacid is a mixture of sodium bicarbonate and magnesium hydroxide, wherein the sodium bicarbonate and magnesium hydroxide are each present in about 0.1 mEq/mg proton pump inhibitor to about 5 mEq/mg proton pump inhibitor. The term “soluble antacid” as used herein refers to an antacid that has a solubility of at least 500 mg/mL, or 300 mg/mL, or 200 mg/mL, or 100 mL/mL in the gastrointestinal fluid.

In some embodiments of the present invention, the antacid is a specific particle size. For example, the average particle size of the antacid may be no greater than 20 μm, or no greater than 30 μm, or no greater than 40 μm, or no greater than 50 μm, or no greater than 60 μm, or no greater than 70 μm, or no greater than 80 μm, or no greater than 90 μm or no greater than 100 μm in diameter. In various embodiments, at least about 70% of the antacid is no greater than 20 μm, or no greater than 30 μm, or no greater than 40 μm, or no greater than 50 μm, or no greater than 60 μm, or no greater than 70 μm, or no greater than 80 μm, or no greater than 90 μm or no greater than 100 μm in diameter. In other embodiments, at least about 85% of the antacid is no greater than 20 μm, or no greater than 30 μm, or no greater than 40 μm, or no greater than 50 μm, or no greater than 60 μm, or no greater than 70 μm, or no greater than 80 μm, or no greater than 90 μm or no greater than 100 μm in diameter.

Shelf-life Enhancing Materials

Materials useful for enhancing the shelf-life of the pharmaceutical formulations of the present invention include materials compatible with the proton pump inhibitor of the pharmaceutical formulations which sufficiently isolate the proton pump inhibitor from other non-compatible excipients. Materials compatible with the proton pump inhibitors of the present invention are those that enhance the shelf-life of the proton pump inhibitor, i.e., by slowing or stopping degradation of the proton pump inhibitor.

Exemplary microencapsulation materials useful for enhancing the shelf-life of pharmaceutical formulations comprising a proton pump inhibitor include, e.g., cellulose hydroxypropyl ethers (HPC) such as EF Klucel®, Nisso HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aqualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials. In other embodiments, the microencapsulation material is selected from hydroxypropylcellulose and cellulose ethers. In still other embodiments, the microencapsulation material is selected from Klucel EF, Klucel EXF, Methocel E5, Methocel E15, and Methocel A15. In other embodiments, the material that enhances the shelf-life has a viscosity of 100-800 cps at 10% solution; or a viscosity of 200-600 cps at 10% solution; or a viscosity of 300-400 cps at 10% solution.

In various embodiments, a buffering agent such as sodium bicarbonate is incorporated into the microencapsulation material. In other embodiments, an antioxidant such as BHT or BHA is incorporated into the microencapsulation material. In still other embodiments, plasticizers such as polyethylene glycols, e.g., PEG 300, PEG 400, PEG 600, PEG 1450, PEG 3350, and PEG 800, stearic acid, propylene glycol, oleic acid, and triacetin are incorporated into the microencapsulation material. In other embodiments, the microencapsulating material useful for enhancing the shelf-life of the pharmaceutical formulations is from the USP or the National Formulary (NF).

In further embodiments, one or more other compatible materials are present in the microencapsulation material. Exemplary materials include, e.g., parietal cell activators, organic solvents, erosion facilitators, diffusion facilitators, anti-adherents, anti-foaming agents, antioxidants, sweetening agents, flavoring agents, and carrier materials such as binders, suspending agents, disintegration agents, filing agents, surfactants, solubilizers, stabilizers, lubricants, wetting agents, and diluents.

A pharmaceutical formulation of the present invention may have an enhanced shelf-life stability if, e.g., the microencapsulated proton pump inhibitor has less than about 0.5% degradation after one month of storage at room temperature, or less than about 1% degradation after one month at room temperature, or less than about 1.5% degradation after one month of storage at room temperature, or less than about 2% degradation after one month storage at room temperature, or less than about 2.5% degradation after one month of storage at room temperature, or less than about 3% degradation after one month of storage at room temperature.

In other embodiments, a pharmaceutical formulation of the present invention may have an enhanced shelf-life stability if the pharmaceutical formulation contains less than about 5% total impurities after about 3 years of storage, or after about 2.5 years of storage, or about 2 years of storage, or about 1.5 years of storage, or about 1 year of storage, or after 11 months of storage, or after 10 months of storage, or after 9 months of storage, or after 8 months of storage, or after 7 months of storage, or after 6 months of storage, or after 5 months of storage, or after 4 months of storage, or after 3 months of storage, or after 2 months of storage, or after 1 month of storage.

In further embodiments, pharmaceutical formulations of the present invention may have enhanced shelf-life stability if the pharmaceutical formulation contains less degradation of the proton pump inhibitor than proton pump inhibitor in the same formulation which is not microencapsulated, or “bare”. For example, if bare proton pump inhibitor in the pharmaceutical formulation degrades at room temperature by more than about 2% after one month of storage and the microencapsulated material degrades at room temperature by less than about 2% after one month of storage, then the proton pump inhibitor has been microencapsulated with a compatible material that enhances the shelf-life of the pharmaceutical formulation.

In some embodiments, the microencapsulating material useful for enhancing the shelf-life of the pharmaceutical formulations increases the shelf-life stability of the pharmaceutical formulation for at least about 5 days at room temperature, or at least about 10 days at room temperature, or at least about 15 days at room temperature, or at least about 20 days at room temperature, or at least about 25 days at room temperature, or at least about 30 days at room temperature or at least about 2 months at room temperature, or at least about 3 months at room temperature, or at least about 4 months at room temperature, or at least about 5 months at room temperature, or at least about 6 months at room temperature, or at least about 7 months at room temperature, or at least about 8 months at room temperature, or at least about 9 months at room temperature, or at least about 10 months at room temperature, or at least about 11 months at room temperature, or at least about one year at room temperature, or at least about 1.5 years at room temperature, or at least about 2 years at room temperature, or at least about 2.5 years at room temperature, or about 3 years at room temperature.

In some embodiments of the present invention, the final formulation of the pharmaceutical formulation will be in the form of a tablet and at least about 50%, or at least about 55%, or at least about 60%, or at least about 65%, or at least about 70%, or at least about 75%, or at least about 80%, or at least about 85% or at least about 90%, or at least about 92%, or at least about 95%, or at least about 98%, or at least about 99% of the microspheres survive the tableting process, wherein microspheres that have survived the tableting process are those which provide the desired properties described herein.

In other embodiments, the final formulation of the pharmaceutical formulation is in the form of a powder for oral suspension and the microencapsulation material surrounding the proton pump inhibitor will sufficiently dissolve in water, with or without stirring, in less than 1 hour, or less than 50 minutes, or less than 40 minutes, or less than 30 minutes, or less than 25 minutes, or less than 20 minutes, or less than 15 minutes, or less than 10 minutes or less than 5 minutes, or less than 1 minute. Sufficiently dissolves means that at least about 50% of the encapsulation material has dissolved.

In various embodiments the microencapsulating material useful for enhancing the shelf-life of the pharmaceutical formulation sufficiently disintegrates to release the proton pump inhibitor into the gastrointestinal fluid of the stomach within less than about 1.5 hours, or within about 10 minutes, or within about 20 minutes, or within about 30 minutes, or within about or within about 40 minutes, or within about 50 minutes, or within about 1 hour, or within about 1.25 hours, or within about 1.5 hours after exposure to the gastrointestinal fluid. Sufficiently disintegrates means that at least about 50% of the microencapsulation material has dissolved.

Taste-masking Materials

Proton pump inhibitors are inherently bitter tasting and in one embodiment of the present invention, these bitter proton pump inhibitors are microencapsulated with a taste-masking material. Materials useful for masking the taste of pharmaceutical formulations include those materials capable of microencapsulating the proton pump inhibitor, thereby protecting the senses from its bitter taste. Taste-masking materials of the present invention provide superior pharmaceutical formulations by e.g., creating a more palatable pharmaceutical formulation as compared to pharmaceutical formulations and/or by creating a dosage form requiring less of the traditional flavoring or tastemasking agents.

The “flavor leadership” criteria used to develop a palatable product include (1) immediate impact of identifying flavor, (2) rapid development of balanced, full flavor, (3) compatible mouth feel factors, (4) no “off” flavors, and (5) short aftertaste. See, e.g., Worthington, A Matter of Taste, Pharmaceutical Executive (April 2001). The pharmaceutical formulations of the present invention improve upon one or more of these criteria.

There are a number of known methods to determine the effect of a taste-masking material such as discrimination tests for testing differences between samples and for ranking a series of samples in order of a specific characteristic; scaling tests used for scoring the specific product attributes such as flavor and appearance; expert tasters used to both quantitatively and qualitatively evaluate a specific sample; affective tests for either measuring the response between two products, measuring the degree of like or dislike of a product or specific attribute, or determine the appropriateness of a specific attribute; and descriptive methods used in flavor profiling to provide objective description of a product are all methods used in the field.

Different sensory qualities of a pharmaceutical formulation such as aroma, flavor, character notes, and aftertaste can be measured using tests know in the art. See, e.g., Roy et al., Modifying Bitterness: Mechanism, Ingredients, and Applications (1997). For example, aftertaste of a product can be measured by using a time vs. intensity sensory measurement. And recently, modern assays have been developed to alert a processor of formulations to the bitter taste of certain substances. Using information known to one of ordinary skill in the art, one would readily be able to determine whether one or more sensory qualities of a pharmaceutical formulation of the present invention have been improved by the use of the taste-masking material.

Taste of a pharmaceutical formulation is important for both increasing patient compliance as well as for competing with other marketed products used for similar diseases, conditions and disorders. Taste, especially bitterness, is particularly important in pharmaceutical formulations for children since, because they cannot weigh the positive benefit of getting better against the immediate negative impact of the bitter taste in their mouth, they are more likely to refuse a drug that tastes bad. Thus, for pharmaceutical formulations for children, it becomes even more important to mask the bitter taste.

Microencapsulation of the proton pump inhibitor can (1) lower the amount of flavoring agents necessary to create a palatable product and/or (2) mask the bitter taste of the proton pump inhibitor by separating the drug from the taste receptors.

Taste-masking materials include, e.g., cellulose hydroxypropyl ethers (HPC) such as Klucel®, Nisswo HPC and PrimaFlo HP22; low-substituted hydroxypropyl ethers (L-HPC); cellulose hydroxypropyl methyl ethers (HPMC) such as Seppifilm-LC, Pharmacoat®, Metolose SR, Opadry YS, PrimaFlo, MP3295A, Benecel MP824, and Benecel MP843; methylcellulose polymers such as Methocel® and Metolose®; Ethylcelluloses (EC) and mixtures thereof such as E461, Ethocel®, Aqualon®-EC, Surelease; Polyvinyl alcohol (PVA) such as Opadry AMB; hydroxyethylcelluloses such as Natrosol®; carboxymethylcelluloses and salts of carboxymethylcelluloses (CMC) such as Aualon®-CMC; polyvinyl alcohol and polyethylene glycol co-polymers such as Kollicoat IR®; monoglycerides (Myverol), triglycerides (KLX), polyethylene glycols, modified food starch, acrylic polymers and mixtures of acrylic polymers with cellulose ethers such as Eudragit® EPO, Eudragit® RD100, and Eudragit® E100; cellulose acetate phthalate; sepifilms such as mixtures of HPMC and stearic acid, cyclodextrins, and mixtures of these materials.

In other embodiments of the present invention, additional taste-masking materials contemplated are those described in U.S. Pat. Nos. 4,851,226, 5,075,114, and 5,876,759. For further examples of taste-masking materials, see, e.g., Remington: The Science and Practice of Pharmacy, Nineteenth Ed. (Easton, Pa.: Mack Publishing Company, 1995); Hoover, John E., Remington 's Pharmaceutical Sciences (Mack Publishing Co., Easton, Pa. 1975); Liberman, H. A. and Lachman, L., Eds., Pharmaceutical Dosage Forms (Marcel Decker, New York, N.Y., 1980); and Pharmaceutical Dosage Forms and Drug Delivery Systems, Seventh Ed. (Lippincott Williams & Wilkins 1999).

In various embodiments, a pH modifier such as sodium carbonate or sodium bicarbonate is incorporated into the microencapsulation material. In other embodiments, an antioxidant such as BHT or BHA is incorporated into the microencapsulation material. In yet another embodiment, sucrose or sucralose is incorporated into the taste masking material. In still other embodiments, plasticizers such as polyethylene glycol and/or stearic acid are incorporated into the microencapsulation material.

In further embodiments, one or more other compatible materials are present in the microencapsulation material. Exemplary materials include, e.g., parietal cell activators, organic solvents, erosion facilitators, diffusion facilitators, anti-adherents, anti-foaming agents, antioxidants, flavoring agents, and carrier materials such as binders, suspending agents, disintegration agents, filing agents, surfactants, solubilizers, stabilizers, lubricants, wetting agents, diluents.

In addition to microencapsulating the proton pump inhibitors with a taste-masking material as described herein, the pharmaceutical formulations of the present invention may also comprise one or more flavoring agents.

“Flavoring agents” or “sweeteners” useful in the pharmaceutical formulations of the present invention include, e.g., acacia syrup, acesulfame K, alitame, anise, apple, aspartame, banana, Bavarian cream, berry, black currant, butterscotch, calcium citrate, camphor, caramel, cherry, cherry cream, chocolate, cinnamon, bubble gum, citrus, citrus punch, citrus cream, cotton candy, cocoa, cola, cool cherry, cool citrus, cyclamate, cylamate, dextrose, eucalyptus, eugenol, fructose, fruit punch, ginger, glycyrrhetinate, glycyrrhiza (licorice) syrup, grape, grapefruit, honey, isomalt, lemon, lime, lemon cream, monoammonium glyrrhizinate (MagnaSweet®), maltol, mannitol, maple, marshmallow, menthol, mint cream, mixed berry, neohesperidine DC, neotame, orange, pear, peach, peppermint, peppermint cream, Prosweet® Powder, raspberry, root beer, rum, saccharin, safrole, sorbitol, spearmint, spearmint cream, strawberry, strawberry cream, stevia, sucralose, sucrose, sodium saccharin, saccharin, aspartame, acesulfame potassium, mannitol, talin, sylitol, sucralose, sorbitol, swiss cream, tagatose, tangerine, thaumatin, tutti fruitti, vanilla, walnut, watermelon, wild cherry, wintergreen, xylitol, or any combination of these flavoring ingredients, e.g., anise-menthol, cherry-anise, cinnamon-orange, cherry-cinnamon, chocolate-mint, honey-lemon, lemon-lime, lemon-mint, menthol-eucalyptus, orange-cream, vanilla-mint, and mixtures thereof. In other embodiments, sodium chloride is incorporated into the pharmaceutical formulation.

Based on the proton pump inhibitor, antacid, and excipients, as well as the amounts of each one, one of skill in the art would be able to determine the best combination of flavors to provide the optimally flavored product for consumer demand and compliance. See, e.g., Roy et al., Modifying Bitterness: Mechanism, Ingredients, and Applications (1997).

In one embodiment, one or more flavoring agents are mixed with the taste-masking material prior to microencapsulating the proton pump inhibitor and, as such, are part of the taste-masking material. In other embodiments, the flavoring agent is mixed with the non-compatible excipients during the formulation process and is therefore not in contact with the proton pump inhibitor, and not part of the microencapsulation material.

In another embodiment, an antacid, such as sodium bicarbonate, is also microencapsulated with one or more taste-masking materials.

In another embodiment, the weight fraction of the taste masking material is, e.g., about 98% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the pharmaceutical composition.

In other embodiments of the present invention, the amount of flavoring agent necessary to create a palatable product, as compared to a pharmaceutical formulation comprising non-microencapsulated proton pump inhibitor, is decreased by 5% or less, or by 5% to 10%, or by 10% to 20%, or by 20% to 30%, or by 30% to 40%, or by 40% to 50%, or by 50% to 60%, or by 60% to 70%, or by 70% to 80%, or by 80% to 90%, or by 90% to 95%, or by greater than 95%. In still other embodiments, no flavoring agent is necessary to create a more palatable pharmaceutical formulation as compared to a similar pharmaceutical formulation comprising non-microencapsulated proton pump inhibitor.

In various embodiments of the invention, the total amount of flavoring agent present in the pharmaceutical formulation is less than 20 grams, or less than 15 grams, or less than 10 grams, or less than 8 grams, or less than 5 grams, or less than 4 grams, or less than 3.5 grams, or less than 3 grams, or less than 2.5 grams or less than 2 grams, or less than 1.5 grams, or less than 1 gram, or less than 500 mg, or less than 250 mg, or less than 150 mg, or less than 100 mg, or less than 50 mg.

Methods of Microencapsulation

The proton pump inhibitor may be microencapsulated by methods known by one of ordinary skill in the art. Such known methods include, e.g., spray drying processes, spinning disk processes, hot melt processes, spray chilling methods, fluidized bed, electrostatic deposition, centrifugal extrusion, rotational suspension separation, polymerization at liquid-gas or solid-gas interface, pressure extrusion, or spraying solvent extraction bath. In addition to these, several chemical techniques, e.g., complex coacervation, solvent evaporation, polymer-polymer incompatibility, interfacial polymerization in liquid media, in situ polymerization, in-liquid drying, and desolvation in liquid media could also be used.

The spinning disk method allows for: 1) an increased production rate due to higher feed rates and use of higher solids loading in feed solution, 2) the production of more spherical particles, 3) the production of a more even coating, and 4) limited clogging of the spray nozzle during the process.

Spray drying is often more readily available for scale-up. In various embodiments, the material used in the spray-dry encapsulation process is emulsified or dispersed into the core material in a concentrated form, e.g., 40-60% solids. The microencapsulation material is, in one embodiment, is emulsified until about 1 to 3 μm droplets are obtained. Once a dispersion of proton pump inhibitor and encapsulation material are obtained, the emulsion is fed as droplets into the heated chamber of the pray drier. In some embodiments, the droplets are sprayed into the chamber or spun off a rotating disk. The microspheres are then dried in the heated chamber and fall to the bottom of the spray drying chamber where they are harvested.

In some embodiments of the present invention, the microspheres have irregular geometries. In other embodiments, the microspheres are aggregates of smaller particles.

In various embodiments, the drug loading of the proton pump inhibitor in the microspheres is greater than 1%, greater than 2.5%, greater than 5%, greater than 10%, greater than 15%, greater than 20%, greater than 25%, greater than 30%, greater than 35%, greater than 40%, greater than 45%, greater than 50%, greater than 55%, greater than 60%, greater than 65%, greater than 70%, greater than 75%, greater than 80% weight percent of the proton pump inhibitor to the microencapsulated drug.

Dosage

The proton pump inhibiting agent is administered and dosed in accordance with good medical practice, taking into account the clinical condition of the individual patient, the site and method of administration, scheduling of administration, and other factors known to medical practitioners. In human therapy, it is important to provide a dosage form that delivers the required therapeutic amount of the drug in vivo, and renders the drug bioavailable in a rapid manner. In addition to the dosage forms described herein, the dosage forms described by Phillips et al. in U.S. Pat. No. 6,489,346 are incorporated herein by reference.

The percent of intact drug that is absorbed into the bloodstream is not narrowly critical, as long as a therapeutic-disorder-effective amount, e.g., a gastrointestinal-disorder-effective amount of a proton pump inhibiting agent, is absorbed following administration of the pharmaceutical composition to a subject. It is understood that the amount of proton pump inhibiting agent and/or antacid that is administered to a subject is dependent on, e.g., the sex, general health, diet, and/or body weight of the subject.

Illustratively, administration of a substituted bicyclic aryl-imidazole to a young child or a small animal, such as a dog, a relatively low amount of the proton pump inhibitor, e.g., about 1 mg to about 30 mg, will often provide blood serum concentrations consistent with therapeutic effectiveness. Where the subject is an adult human or a large animal, such as a horse, achievement of a therapeutically effective blood serum concentration will require larger dosage units, e.g., about 10 mg, about 15 mg, about 20 mg, about 30 mg, about 40 mg, about 80 mg, or about 120 mg dose for an adult human, or about 150 mg, or about 200 mg, or about 400 mg, or about 800 mg, or about 1000 mg dose, or about 1500 mg dose, or about 2000 mg dose, or about 2500 mg dose, or about 3000 mg dose, or about 3200 mg dose, or about 3500 mg dose for an adult horse.

In various other embodiments of the present invention, the amount of proton pump inhibitor administered to a subject is, e.g., about 1-2 mg/Kg of body weight, or about 0.5 mg/Kg of body weight, or about 1 mg/Kg of body weight, or about 1.5 mg/Kg of body weight, or about 2 mg/Kg of body weight.

Treatment dosages generally may be titrated to optimize safety and efficacy. Typically, dosage-effect relationships from in vitro and/or in vivo tests initially can provide useful guidance on the proper doses for subject administration. Studies in animal models generally may be used for guidance regarding effective dosages for treatment of gastrointestinal disorders or diseases in accordance with the present invention. In terms of treatment protocols, it should be appreciated that the dosage to be administered will depend on several factors, including the particular agent that is administered, the route chosen for administration, the condition of the particular subject.

In various embodiments, unit dosage forms for humans contain about 1 mg to about 120 mg, or about 1 mg, or about 5 mg, or about 10 mg, or about 15 mg, or about 20 mg, or about 30 mg, or about 40 mg, or about 50 mg, or about 60 mg, or about 70 mg, or about 80, mg, or about 90 mg, or about 100 mg, or about 110 mg, or about 120 mg of a proton pump inhibitor.

In a further embodiment of the present invention, the pharmaceutical formulation is administered in an amount to achieve a measurable serum concentration of a non-acid degraded proton pump inhibiting agent greater than about 100 ng/ml within about 30 minutes after administration of the pharmaceutical formulation. In another embodiment of the present invention, the pharmaceutical formulation is administered to the subject in an amount to achieve a measurable serum concentration of a non-acid degraded or non-acid reacted proton pump inhibiting agent greater than about 100 ng/ml within about 15 minutes after administration of the pharmaceutical formulation. In yet another embodiment, the pharmaceutical formulation is administered to the subject in an amount to achieve a measurable serum concentration of a non-acid degraded or non-acid reacted proton pump inhibiting agent greater than about 100 ng/ml within about 10 minutes after administration of the pharmaceutical formulation.

In another embodiment of the present invention, the composition is administered to the subject in an amount to achieve a measurable serum concentration of the proton pump inhibiting agent greater than about 150 ng/ml within about 15 minutes and to maintain a serum concentration of the proton pump inhibiting agent of greater than about 150 ng/ml from about 15 minutes to about 1 hour after administration of the composition. In yet another embodiment of the present invention, the composition is administered to the subject in an amount to achieve a measurable serum concentration of the proton pump inhibiting agent greater than about 250 ng/ml within about minutes and to maintain a serum concentration of the proton pump inhibiting agent of greater than about 150 ng/ml from about 15 minutes to about 1 hour after administration of the composition. In another embodiment of the present invention, the composition is administered to the subject in an amount to achieve a measurable serum concentration of the proton pump inhibiting agent greater than about 350 ng/ml within about 15 minutes and to maintain a serum concentration of the proton pump inhibiting agent of greater than about 150 ng/ml from about 15 minutes to about 1 hour after administration of the composition. In another embodiment of the present invention, the composition is administered to the subject in an amount to achieve a measurable serum concentration of the proton pump inhibiting agent greater than about 450 ng/ml within about 15 minutes and to maintain a serum concentration of the proton pump inhibiting agent of greater than about 150 ng/ml from about 15 minutes to about 1 hour after administration of the composition.

In another embodiment of the present invention, the composition is administered to the subject in an amount to achieve a measurable serum concentration of the proton pump inhibiting agent greater than about 150 ng/ml within about 30 minutes and to maintain a serum concentration of the proton pump inhibiting agent of greater than about 150 ng/ml from about 30 minutes to about 1 hour after administration of the composition. In yet another embodiment of the present invention, the composition is administered to the subject in an amount to achieve a measurable serum concentration of the proton pump inhibiting agent greater than about 250 ng/ml within about 30 minutes and to maintain a serum concentration of the proton pump inhibiting agent of greater than about 150 ng/ml from about 30 minutes to about 1 hour after administration of the composition. In another embodiment of the present invention, the composition is administered to the subject in an amount to achieve a measurable serum concentration of the proton pump inhibiting agent greater than about 350 ng/ml within about 30 minutes and to maintain a serum concentration of the proton pump inhibiting agent of greater than about 150 ng/ml from about 30 minutes to about 1 hour after administration of the composition. In another embodiment of the present invention, the composition is administered to the subject in an amount to achieve a measurable serum concentration of the proton pump inhibiting agent greater than about 450 ng/ml within about 30 minutes and to maintain a serum concentration of the proton pump inhibiting agent of greater than about 150 ng/ml from about 30 minutes to about 1 hour after administration of the composition.

In still another embodiment of the present invention, the composition is administered to the subject in an amount to achieve a measurable serum concentration of a non-acid degraded or non-acid reacted proton pump inhibiting agent greater than about 500 ng/ml within about 1 hour after administration of the composition. In yet another embodiment of the present invention, the composition is administered to the subject in an amount to achieve a measurable serum concentration of a non-acid degraded or non-acid reacted proton pump inhibiting agent greater than about 300 ng/ml within about 45 minutes after administration of the composition.

Contemplated compositions of the present invention provide a therapeutic effect as proton pump inhibiting agent medications over an interval of about 5 minutes to about 24 hours after administration, enabling, for example, once-a-day, twice-a-day, three times a day, etc. administration if desired.

Generally speaking, one will desire to administer an amount of the compound that is effective to achieve a serum level commensurate with the concentrations found to be effective in vivo for a period of time effective to elicit a therapeutic effect. Determination of these parameters is well within the skill of the art. These considerations are well known in the art and are described in standard textbooks.

In one embodiment of the present invention, the composition is administered to a subject in a gastrointestinal-disorder-effective amount, that is, the composition is administered in an amount that achieves a therapeutically-effective dose of a proton pump inhibiting agent in the blood serum of a subject for a period of time to elicit a desired therapeutic effect. Illustratively, in a fasting adult human (fasting for generally at least 10 hours) the composition is administered to achieve a therapeutically-effective dose of a proton pump inhibiting agent in the blood serum of a subject within about 45 minutes after administration of the composition. In another embodiment of the present invention, a therapeutically-effective dose of the proton pump inhibiting agent is achieved in the blood serum of a subject within about 30 minutes from the time of administration of the composition to the subject. In yet another embodiment, a therapeutically-effective dose of the proton pump inhibiting agent is achieved in the blood serum of a subject within about 20 minutes from the time of administration to the subject. In still another embodiment of the present invention, a therapeutically-effective dose of the proton pump inhibiting agent is achieved in the blood serum of a subject at about 15 minutes from the time of administration of the composition to the subject.

In further embodiments, greater than about 98%; or greater than about 95%; or greater than about 90%; or greater than about 75%; or greater than about 50% of the drug absorbed into the bloodstream is in a non-acid degraded or a non-acid reacted form.

In other embodiments, the pharmaceutical formulations provide a release profile of the proton pump inhibitor, using USP dissolution methods, whereby greater than about 50% of the proton pump inhibitor is released from the composition within about 2 hours; or greater than 50% of the proton pump inhibitor is released from the composition within about 1.5 hours; or greater than 50% of the proton pump inhibitor is released from the composition within about 1 hour after exposure to gastrointestinal fluid. In another embodiment, greater than about 60% of the proton pump inhibitor is released from the composition within about 2 hours; or greater than 60% of the proton pump inhibitor is released from the composition within about 1.5 hours; or greater than 60% of the proton pump inhibitor is released from the composition within about 1 hour after exposure to gastrointestinal fluid. In yet another embodiment, greater than about 70% of the proton pump inhibitor is released from the composition within about 2 hours; or greater than 70% of the proton pump inhibitor is released from the composition within about 1.5 hours; or greater than 70% of the proton pump inhibitor is released from the composition within about 1 hour after exposure to gastrointestinal fluid.

Pharmaceutical Compositions

The pharmaceutical formulations of the present invention contain desired amounts of microencapsulated proton pump inhibitor and antacid and can be in the form of, e.g., a tablet; including a suspension tablet, a chewable tablet, or an effervescent tablet; a pill; a powder such as a sterile packaged powder, a dispensable powder, and an effervescent powder; a capsule including both soft or hard gelatin capsules such as HPMC capsules; a lozenge; a sachet; a troche; pellets; granules; or aerosol. These pharmaceutical formulations of the present invention can be manufactured by conventional pharmacological techniques.

Conventional pharmacological techniques include, e.g., one or a combination of methods: (1) dry mixing, (2) direct compression, (3) milling, (4) dry or non-aqueous granulation, (5) wet granulation, or (6) fusion. See, e.g., Lachman et al., The Theory and Practice of Industrial Pharmacy (1986). Other methods include, e.g., prilling, spray drying, pan coating, melt granulation, granulation, wurster coating, tangential coating, top spraying, tableting, extruding, coacervation and the like.

In one embodiment, the proton pump inhibitor is microencapsulated prior to being formulated into one of the above forms. In another embodiment, some or all of the antacid is also microencapsulated prior to being further formulated into one of the above forms. In still other embodiments, using standard coating procedures, such as those described in Remington's Pharmaceutical Sciences, 20th Edition (2000), a film coating is provided around the pharmaceutical formulation.

Provided herein are pharmaceutical formulations wherein some or all of the proton pump inhibitor and some or all of the antacid are microencapsulated. In some embodiments, only some of the proton pump inhibitor is microencapsulated. In other embodiments, all of the proton pump inhibitor is microencapsulated. In still other embodiments, only some of the antacid is microencapsulated.

In various embodiments, the average particle sizes of the microencapsulated drugs range from submicron to less than about 1,000 microns in diameter, or less than about 900 microns in diameter, or less than about 800 microns in diameter, or less than about 700 microns in diameter, or less than about 600 microns in diameter, or less than about 500 microns in diameter, or less than about 450 microns in diameter, or less than about 400 microns in diameter, or less than about 350 microns in diameter, or less than about 300 microns in diameter, or less than about 250 microns in diameter, or less than about 200 microns in diameter, or less than about 150 microns in diameter, or less than about 100 microns in diameter, or less than about 75 microns in diameter, or less than about 50 microns in diameter, or less than about 25 microns in diameter, or less than about 15 microns in diameter. In other embodiments, the average particle size of the aggregates is between about 25 microns in diameter to about 300 microns in diameter. In still other embodiments, the average particle size of the aggregates is between about 100 microns in diameter to about 200 microns in diameter. And in still further embodiments, the average particle size of the aggregates is between about 25 microns in diameter to about 100 microns in diameter. The term “average particle size” is intended to describe the average diameter of the particles and/or agglomerates used in the pharmaceutical formulation.

In other embodiments, the pharmaceutical formulations further comprise one or more additional materials such as a pharmaceutically compatible carrier, binder, filling agent, suspending agent, flavoring agent, sweetening agent, disintegrating agent, surfactant, preservative, lubricant, colorant, diluent, solubilizer, moistening agent, stabilizer, wetting agent, anti-adherent, parietal cell activator, anti-foaming agent, antioxidant, chelating agent, antifungal agent, antibacterial agent, or one or more combination thereof.

Parietal cell activators are administered in an amount sufficient to produce the desired stimulatory effect without causing untoward side effects to patients. In one embodiment, the parietal cell activator is administered in an amount of about 5 mg to about 2.5 grams per 20 mg dose of the proton pump inhibitor.

In other embodiments, one or more layers of the pharmaceutical formulation are plasticized. Illustratively, a plasticizer is generally a high boiling point solid or liquid. Suitable plasticizers can be added from about 0.01% to about 50% by weight (w/w) of the coating composition. Plasticizers include, e.g., diethyl phthalate, citrate esters, polyethylene glycol, glycerol, acetylated glycerides, triacetin, polypropylene glycol, polyethylene glycol, triethyl citrate, dibutyl sebacate, stearic acid, stearol, stearate, and castor oil.

Exemplary Solid Compositions

Solid compositions, e.g., tablets, chewable tablets, effervescent tablets, and capsules, are prepared by mixing the microencapsulated proton pump inhibitor with one or more antacid and pharmaceutical excipients to form a bulk blend composition. When referring to these bulk blend compositions as homogeneous, it is meant that the microencapsulated proton pump inhibitor and antacid are dispersed evenly throughout the composition so that the composition may be readily subdivided into equally effective unit dosage forms, such as tablets, pills, and capsules. The individual unit dosages may also comprise film coatings, which disintegrate upon oral ingestion or upon contact with diluent.

Compressed tablets are solid dosage forms prepared by compacting the bulk blend compositions described above. In various embodiments, compressed tablets of the present invention will comprise one or more flavoring agents. In other embodiments, the compressed tablets will comprise a film surrounding the final compressed tablet. In other embodiments, the compressed tablets comprise one or more excipients and/or flavoring agents.

A capsule may be prepared, e.g., by placing the bulk blend composition, described above, inside of a capsule.

A chewable tablet may be prepared by compacting bulk blend compositions, described above. In one embodiment, the chewable tablet comprises a material useful for enhancing the shelf-life of the pharmaceutical formulation. In another embodiment, microencapsulated material has taste-masking properties. In various other embodiments, the chewable tablet comprises one or more flavoring agents and one ore more taste-masking materials. In yet other embodiments the chewable tablet comprises both a material useful for enhancing the shelf-life of the pharmaceutical formulation and one or more flavoring agents.

In various embodiments, the microencapsulated proton pump inhibitor, antacid, and optionally one or more excipients are dry blended and compressed into a mass, such as a tablet, having a hardness sufficient to provide a pharmaceutical composition that substantially disintegrates within less than about 30 minutes, less than about 35 minutes, less than about 40 minutes, less than about 45 minutes, less than about 50 minutes, less than about 55 minutes, or less than about 60 minutes, after oral administration, thereby releasing the antacid and the proton pump inhibitor into the gastrointestinal fluid. When at least 50% of the pharmaceutical composition has disintegrated, the compressed mass has substantially disintegrated.

Exemplary Powder Compositions

A powder for suspension may be prepared by combining microencapsulated proton pump inhibitor and one or more antacid. In various embodiments, the powder may comprise one or more pharmaceutical excipients. In some embodiments, the proton pump inhibitor is micronized. Other embodiments of the present invention also comprise a suspending agent and/or a wetting agent.

Effervescent powders are also prepared in accordance with the present invention. Effervescent salts have been used to disperse medicines in water for oral administration. Effervescent salts are granules or coarse powders containing a medicinal agent in a dry mixture, usually composed of sodium bicarbonate, citric acid and/or tartaric acid. When salts of the present invention are added to water, the acids and the base react to liberate carbon dioxide gas, thereby causing “effervescence.” Examples of effervescent salts include the following ingredients: sodium bicarbonate or a mixture of sodium bicarbonate and sodium carbonate, citric acid and/or tartaric acid. Any acid-base combination that results in the liberation of carbon dioxide can be used in place of the combination of sodium bicarbonate and citric and tartaric acids, as long as the ingredients were suitable for pharmaceutical use and result in a pH of about 6 or higher.

The method of preparation of the effervescent granules of the present invention employs three basic processes: wet granulation, dry granulation and fusion. The fusion method is used for the preparation of most commercial effervescent powders. It should be noted that, although these methods are intended for the preparation of granules, the formulations of effervescent salts of the present invention could also be prepared as tablets, according to known technology for tablet preparation.

Wet granulation is one the oldest method of granule preparation. The individual steps in the wet granulation process of tablet preparation include milling and sieving of the ingredients, dry powder mixing, wet massing, granulation, and final grinding. In various embodiments, the microencapsulated omeprazole is added to the other excipients of the pharmaceutical formulation after they have been wet granulated.

Dry granulation involves compressing a powder mixture into a rough tablet or “slug” on a heavy-duty rotary tablet press. The slugs are then broken up into granular particles by a grinding operation, usually by passage through an oscillation granulator. The individual steps include mixing of the powders, compressing (slugging) and grinding (slug reduction or granulation). No wet binder or moisture is involved in any of the steps. In some embodiments, the microencapsulated omeprazole is dry granulated with other excipients in the pharmaceutical formulation. In other embodiments, the microencapsulated omeprazole is added to other excipients of the pharmaceutical formulation after they have been dry granulated.

Other Exemplary Compositions

Pharmaceutical compositions suitable for buccal (sublingual) administration include, e.g., lozenges in a flavored base, such as sucrose, acacia, tragacanth, and pastilles comprising microencapsulated proton pump inhibitor in an inert base such as gelatin, glycerin, sucrose, and acacia.

Many other types of release delivery systems are available and known to those of ordinary skill in the art. Examples of such delivery systems include, e.g., polymer-based systems, such as polylactic and polyglycolic acid, plyanhydrides and polycaprolactone;
nonpolymer-based systems that are lipids, including sterols, such as cholesterol, cholesterol esters and fatty acids, or neutral fats, such as mono-, di- and triglycerides; hydrogel release systems; silastic systems; peptide-based systems; wax coatings; compressed tablets using conventional binders and excipients partially fused implants and the like. See, e.g., Liberman et al., Pharmaceutical Dosage Forms, 2 Ed., Vol. 1, pp. 209-214 (1990).

In some embodiments, the pharmaceutical composition comprises (a) microencapsulated proton pump inhibitor; and (b) at least one antacid; wherein the pharmaceutical composition is made by the process of (a) microencapsulating some or all of the proton pump inhibitor; and (b) dry blending the microencapsulated material with some or all of the at least one antacid. In other embodiments, the pharmaceutical composition comprises (a) microencapsulated proton pump inhibitor, and (b) at least one antacid, wherein the microencapsulated proton pump inhibitor is made by the process of (a) spray drying the proton pump inhibitor with a microencapsulating material. In still other embodiments, the pharmaceutical composition comprises (a) microencapsulated proton pump inhibitor, and (b) at least one antacid, wherein the pharmaceutical composition is made by the process of (a) microencapsulating some or all of the proton pump inhibitor, and (b) blending the microencapsulated material with some or all of the at least one antacid.

Treatment

Initial treatment of a subject suffering from a disease, condition or disorder where treatment with an inhibitor of H⁺/K⁺-ATPase is indicated can begin with the dosages indicated above. Treatment is generally continued as necessary over a period of hours, days, or weeks to several months or years until the disease, condition or disorder has been controlled or eliminated. Subjects undergoing treatment with the compositions disclosed herein can be routinely monitored by any of the methods well known in the art to determine the effectiveness of therapy. Continuous analysis of such data permits modification of the treatment regimen during therapy so that optimal effective amounts of compounds of the present invention are administered at any point in time, and so that the duration of treatment can be determined as well. In this way, the treatment regimen/dosing schedule can be rationally modified over the course of therapy so that the lowest amount of an inhibitor of H⁺/K⁺-ATPase exhibiting satisfactory effectiveness is administered, and so that administration is continued only so long as is necessary to successfully treat the disease, condition or disorder.

In one embodiment, the pharmaceutical formulations are useful for treating a condition, disease or disorder where treatment with a proton pump inhibitor is indicated. In other embodiments, the treatment method comprises oral administration of one or more compositions of the present invention to a subject in need thereof in an amount effective at treating the condition, disease, disorder. In another embodiment, the disease, condition or disorder is a gastrointestinal disorder. The dosage regimen to prevent, give relief from, or ameliorate the disease, condition or disorder can be modified in accordance with a variety of factors. These factors include the type, age, weight, sex, diet, and medical condition of the subject and the severity of the disorder or disease. Thus, the dosage regimen actually employed can vary widely and therefore can deviate from the dosage regimens set forth herein.

In some embodiments, the pharmaceutical formulation is administered post meal. In further embodiments, the pharmaceutical formulation administered post meal is in the form of a chewable tablet.

The present invention also includes methods of treating, preventing, reversing, halting or slowing the progression of a gastrointestinal disorder once it becomes clinically evident, or treating the symptoms associated with, or related to the gastrointestinal disorder, by administering to the subject a composition of the present invention. The subject may already have a gastrointestinal disorder at the time of administration, or be at risk of developing a gastrointestinal disorder. The symptoms or conditions of a gastrointestinal disorder in a subject can be determined by one skilled in the art and are described in standard textbooks. The method comprises the oral administration a gastrointestinal-disorder-effective amount of one or more compositions of the present invention to a subject in need thereof.

Gastrointestinal disorders include, e.g., duodenal ulcer disease, gastrointestinal ulcer disease, gastroesophageal reflux disease, erosive esophagitis, poorly responsive symptomatic gastroesophageal reflux disease, pathological gastrointestinal hypersecretory disease, Zollinger Ellison Syndrome, and acid dyspepsia. In one embodiment of the present invention, the gastrointestinal disorder is heartburn.

Besides being useful for human treatment, the present invention is also useful for other subjects including veterinary animals, reptiles, birds, exotic animals and farm animals, including mammals, rodents, and the like. Mammals include primates, e.g., a monkey, or a lemur, horses, dogs, pigs, or cats. Rodents includes rats, mice, squirrels, or guinea pigs.

In various embodiments of the present invention, the compositions are designed to produce release of the proton pump inhibitor to the site of delivery (typically the stomach), while substantially preventing or inhibiting acid degradation of the proton pump inhibitor.

The present pharmaceutical compositions can also be used in combination (“combination therapy”) with another pharmaceutical agent that is indicated for treating or preventing a gastrointestinal disorder, such as, e.g., an anti-bacterial agent, an alginate, a prokinetic agent, a H2 antagonist, an antacid, or sucralfate, which are commonly administered to minimize the pain and/or complications related to this disorder.

Combination therapies contemplated by the present invention include administration of a pharmaceutical formulation of the present invention in conjunction with another pharmaceutically active agent that is indicated for treating or preventing a gastrointestinal disorder in a subject, as part of a specific treatment regimen intended to provide a beneficial effect from the co-action of these therapeutic agents for the treatment of a gastrointestinal disorder. The beneficial effect of the combination includes, but is not limited to, pharmacokinetic or pharmacodynamic co-action resulting from the combination of therapeutic agents. Administration of these therapeutic agents in combination typically is carried out over a defined time period (usually substantially simultaneously, minutes, hours, days, weeks, months or years depending upon the combination selected).

Combination therapies of the present invention are also intended to embrace administration of these therapeutic agents in a sequential manner, that is, where each therapeutic agent is administered at a different time, as well as administration of these therapeutic agents, or at least two of the therapeutic agents, in a substantially simultaneous manner. Substantially simultaneous administration can be accomplished, e.g., by administering to the subject a single tablet or capsule having a fixed ratio of each therapeutic agent or in multiple, single capsules, or tablets for each of the therapeutic agents. Sequential or substantially simultaneous administration of each therapeutic agent can be effected by any appropriate route.

The composition of the present invention can be administered orally or nasogastrointestinal, while the other therapeutic agent of the combination can be administered by any appropriate route for that particular agent, including, but not limited to, an oral route, a percutaneous route, an intravenous route, an intramuscular route, or by direct absorption through mucous membrane tissues. For example, the composition of the present invention is administered orally or nasogastrointestinal and the therapeutic agent of the combination may be administered orally, or percutaneously. The sequence in which the therapeutic agents are administered is not narrowly critical. Combination therapy also can embrace the administration of the therapeutic agents as described above in further combination with other biologically active ingredients, such as, but not limited to, a pain reliever, such as a steroidal or nonsteroidal anti-inflammatory drug, or an agent for improving stomach motility, e.g., and with non-drug therapies, such as, but not limited to, surgery.

The therapeutic compounds which make up the combination therapy may be a combined dosage form or in separate dosage forms intended for substantially simultaneous administration. The therapeutic compounds that make up the combination therapy may also be administered sequentially, with either therapeutic compound being administered by a regimen calling for two step administration. Thus, a regimen may call for sequential administration of the therapeutic compounds with spaced-apart administration of the separate, active agents. The time period between the multiple administration steps may range from, e.g., a few minutes to several hours to days, depending upon the properties of each therapeutic compound such as potency, solubility, bioavailability, plasma half-life and kinetic profile of the therapeutic compound, as well as depending upon the effect of food ingestion and the age and condition of the subject. Circadian variation of the target molecule concentration may also determine the optimal dose interval.

The therapeutic compounds of the combined therapies contemplated by the present invention, whether administered simultaneously, substantially simultaneously, or sequentially, may involve a regimen calling for administration of one therapeutic compound by oral route and another therapeutic compound by an oral route, a percutaneous route, an intravenous route, an intramuscular route, or by direct absorption through mucous membrane tissues, for example. Whether the therapeutic compounds of the combined therapy are administered orally, by inhalation spray, rectally, topically, buccally, sublingually, or parenterally (e.g., subcutaneous, intramuscular, intravenous and intradermal injections, or infusion techniques), separately or together, each such therapeutic compound will be contained in a suitable pharmaceutical formulation of pharmaceutically-acceptable excipients, diluents or other formulations components.

In one embodiment, the pharmaceutical formulations of the present invention are administered with low strength enteric coated Aspirin. In another embodiment, the second active pharmaceutical, e.g., Aspirin or an NSAID, used in combination with the pharmaceutical formulations of the present invention, is enteric coated. In other embodiments, antacid present in the pharmaceutical formulations of the present invention increase the pH level of the gastrointestinal fluid, thereby allowing part or all of the enteric coating on the second active pharmaceutical to dissolve in the stomach.

For the sake of brevity, all patents and other references cited herein are incorporated by reference in their entirety.

### EXAMPLES

The present invention is further illustrated by the following examples, which should not be construed as limiting in any way. The experimental procedures to generate the data shown are discussed in more detail below. For all formulations herein, multiple doses may be proportionally compounded as is known in the art. The coatings, layers and encapsulations are applied in conventional ways using equipment customary for these purposes.

The invention has been described in an illustrative manner, and it is to be understood that the terminology used is intended to be in the nature of description rather than of limitation.

### Example 1

### Microencapsulation Materials and Methods

Microencapsulation Process Using Spinning Disk Atomization

The basic operation for the spinning disk process used was as follows: An encapsulation solution was prepared by dissolving the encapsulation material in the appropriate solvent. Omeprazole was dispersed in the coating solution and fed onto the center of the spinning disk. A thin film was produced across the surface of the disk and atomization occurs as the coating material left the periphery of the disk. The microspheres were formed by removal of the solvent using heated airflow inside the atomization chamber and collected as a free-flowing powder using a cyclone separator.

Spray Drying Microencapsulation Process

A spray dryer consisted of the same components as a spinning disk except atomization by a high pressure nozzle or two-fluid nozzle instead of a spinning disk can be also used.

A spray dryer with attached fluid-bed dryer for sizing of dried particles and/or agglomeration if desired can be also used. Recycling of the super-fine particles from the cyclones back to the spray dryer inlet would allow the agglomeration to form desired particle size distribution.

The dissolution profiles of the microencapsulated omeprazole were determined by a method similar to the HPLC method outlined in Example 10, described below. The size of the microspheres was determined by using a microscopic optical method similar to the one outlined in Example 11.

| | OME load | | | | | | | |
|---|---|---|---|---|---|---|---|---|
| | (wt %) | | | | | | | |
| | Theoretical/ | | | | % Omeprazole Released (wt %) | | | |
| Sample | Analytical | Material | Method | Size | 5 min | 30 min | 45 min | 2 hour |
| 4 | 25%/22% | KLX | Disk-hot | 25-125 | −1.1 | 10.3 | 22.2 | 36.5 |
| | | BHT (0.1% of | melt | micron | 1.5 | 10.1 | 17.3 | 34.6 |
| | | KLX) | | | | | | |
| 5 | 25%/23% | Methocel | Spray dry | 5-30 | 103.7 | 100.7 | 99.2 | 98.3 |
| | | A15LV PEG | | micron | 113.1 | 103.7 | 102.7 | 101.0 |
| | | 3350 (5%) | | | | | | |
| 6 | 25%/26% | Methocel | Spray dry | 5-30 | 77.5 | 85.6 | 85.0 | 86.1 |
| | | A15LV PEG | | micron | 120.3 | 87.3 | 90.4 | 86.3 |
| | | 300 (5%) | | | | | | |
| | | BHT (0.1%) | | | | | | |
| 7 | 25%/39% | Methocel | Spray dry | 5-30 | 29.8 | 37.7 | 41.5 | 51.7 |
| | | A15LV | | micron | 33.8 | 30.7 | 28.2 | 38.0 |
| | | Span 20 (5%) | | | | | | |
| | | BHT (0.1%) | | | | | | |
| 8 | 25%/24% | Methocel | Spray dry | 5-30 | 89.8 | 97.2 | 95.1 | 90.4 |
| | | A15LV BHT | | micron | 93.1 | 82.5 | 83.9 | 84.8 |
| | | (0.1%) | | | | | | |
| 10 | 3%/2% | Methocel | Spray dry | 5-30 | 94.4 | 104.7 | 102.7 | 97.9 |
| | | A15LV PEG | | micron | 104.6 | 99.2 | 98.2 | 93.2 |
| | | 3350 (5%) | | | | | | |
| | | BHT (0.1%) | | | | | | |
| | | Sodium | | | | | | |
| | | bicarbonate | | | | | | |
| 11 | 25%/20% | Opadry YS-1- | Spray dry | 5-30 | 91.4 | 103.1 | 100.1 | 94.8 |
| | | 7003 PEG | | micron | 99.3 | 98.8 | 95.8 | 91.0 |
| | | 3350 (5%) | | | | | | |
| | | BHT (0.1%) | | | | | | |
| 12 | 25%/27% | Methocel | Spray dry | 5-30 | 134.1 | 92.9 | 86.9 | 85.0 |
| | | K4M PEG | | micron | 73.2 | 88.4 | 85.3 | 84.2 |
| | | 3350 (10%) | | | | 74.5 | 75.3 | 73.5 |
| | | BHT | | | | 78.7 | 77.2 | 74.1 |
| 13 | 25%/26% | Kollicoat IR | Spray dry | 5-30 | 99.1 | 94.7 | 94.2 | 91.9 |
| | | PEG 3350 | | micron | 89.7 | 87.7 | 84.9 | 84.6 |
| | | (5%) | | | | | | |
| | | BHT | | | | | | |
| 14 | 25%/21% | Eudragit RD | Spray dry | 5-30 | 111.5 | 72.6 | 76.9 | 73.0 |
| | | 100 PEG | | micron | 48.9 | 73.1 | 74.1 | 73.7 |
| | | 3350 (5%) | | | | | | |
| | | BHT (0.1%) | | | | | | |
| 15 | 25%/26% | Klucel (HPC) | Spray dry | 5-30 | | 76.8 | 82.1 | 83.1 |
| | | PEG 3350 | | micron | | 69.6 | 71.7 | 73.1 |
| | | (5%) BHT | | | | | | |
| | | (0.1%) | | | | | | |
| 16 | 25%/25% | Ethocel #7 | Disk- | 25-125 | 5.4 | 9.3 | 13.8 | 23.8 |
| | | | solvent | micron | 14 | 9.7 | 12.4 | 22.5 |
| 17 | 25%/25% | Ethocel (50%) | Disk- | 25-125 | 122.6 | 105.9 | 106.2 | 97.6 |
| | | Methocel E5 | solvent | micron | 113.4 | 100.4 | 103.9 | 97.9 |
| | | (50%) | | | | | | |
| 18 | 25%/25% | Ethocel (75%) | Disk- | 25-125 | | 61.6 | 73.0 | 60.9 |
| | | Methocel | solvent | micron | | 44.3 | 53.8 | 67.9 |
| | | (25%) | | | | 37.0 | 47.0 | 59.2 |
| | | | | | | 40.5 | 47.6 | 61.1 |
| 19 | 25%/25% | Methocel | Disk- | 25-125 | | 78.7 | 80.5 | 78.1 |
| | | | solvent | micron | | 84.8 | 84.8 | 78.7 |
| | | | | | | 78.0 | 80.3 | 78.1 |
| | | | | | | 79.0 | 75.2 | 77.0 |
| 20 | 2.4%/5% | Ethocel | Disk- | 25-125 | | 25.0 | 28.8 | 33.6 |
| | | Sodium | solvent | micron, | | 23.2 | 33.3 | 30.3 |
| | | Bicarbonate | | | | 19.3 | 20.6 | 27.4 |
| | | | | | | 16.1 | 17.4 | 22.6 |
| 21 | 25%/22% | Ethocel | Disk- | 25-125 | | 31.7 | 44.6 | 59.4 |
| | | PEG 3350 | solvent | micron | | 38.1 | 52.5 | 59.6 |
| | | (5%) | | | | | | |
| 22 | 25%/22% | Ethocel (50%) | Disk- | 25-125 | | 89.9 | 88.6 | 86.7 |
| | | Klucel EXAF | solvent | micron | | 84.5 | 88.4 | 85.0 |
| | | (50%) | | | | | | |
| 23 | 25%/22% | Klucel | Disk- | 25-100 | | 88.1 | 90.2 | 88.1 |
| | | | solvent | microns | | 83.2 | 82.9 | 82.3 |
| 24 | 25%/22% | Sepifilm LP | Disk- | 25-100 | | 97.0 | 95.2 | 92.2 |
| | | | solvent | micron | | 90.3 | 89.8 | 90.1 |
| 25 | 25%/23% | Eudragit E100 | Disk- | 25-80 | | 13.2 | 17.0 | 24.8 |
| | | | solvent | micron | | 8.2 | 12.1 | 20.2 |
| 26 | 40%/35% | Eudragit E100 | Disk- | 25-80 | | 5.1 | 6.4 | 11.5 |
| | | | solvent | micron | | 13.1 | 16.4 | 23.5 |
| 27 | 40%/38% | Eudragit E100 | Disk- | 25-80 | | 15.0 | 16.2 | 27.0 |
| | | Span 20 (5%) | solvent | micron | | 16.9 | 20.1 | 26.3 |
| 28 | 40%/35% | Eudragit E100 | Disk- | 25-80 | | 16.3 | 19.5 | 28.8 |
| | | PEG 300 (5%) | solvent | micron | | 16.0 | 12.9 | 28.5 |
| 29 | 25%/25% | Eudragit EPO | Disk- | 25-80 | | 15.3 | 17.8 | 25.6 |
| | | | solvent | micron | | 11.9 | 14.5 | 21.2 |
| 30 | 40%/36% | Eudragit EPO | Disk- | 25-90 | | 15.2 | 17.8 | 27.1 |
| | | | solvent | micron | | 17.5 | 17.5 | 30.9 |
| 31 | 25%/24% | Opadry AMB | Spray dry | <30 | | 105.8 | 104.0 | 77.5 |
| | | | | micron | | 105.8 | 103.8 | 98.6 |
| 34 | 25%/23% | Kollicoat IR | Spray dry | | | 99.4 | 94.0 | 83.4 |
| | | | | | | 101.6 | 99.5 | 96.3 |
| 35 | 25%/26% | Kollicoat IR | Spray dry | <30 | | 104.2 | 97.0 | 86.3 |
| | | Sodium | | micron | | 99.1 | 95.3 | 91.1 |
| | | bicarbonate | | | | | | |
| 38 | 25%/26% | Klucel | Spray dry | <30 | | 81.3 | 77.3 | 72.1 |
| | | Sodium | | micron | | 93.8 | 90.5 | 85.8 |
| | | bicarbonate | | | | | | |
| 39 | 25%/15% | Klucel(60%) | Spray dry | <50 | | 91.4 | 86.4 | 82.6 |
| | | Sucraolse | | micron | | 101.5 | 97.2 | 93.4 |
| | | (10%) Sodium | | | | | | |
| | | bicarbonate | | | | | | |
| | | (30%) | | | | | | |
| 40 | 50%/47% | Eudragit EPO | Disk- | 20-75 | | 10.2 | 14.0 | 23.5 |
| | | | solvent | microns | | 10.6 | 13.6 | 24.3 |
| 41 | 60%/57% | Eudragit EPO | Disk- | 20-90 | | 13.9 | 17.5 | 35.7 |
| | | | solvent | microns | | 6.7 | 17.4 | 33.8 |
| 42 | 40%/39% | Eudragit | Disk- | 20-85 | | 17.0 | 20.2 | 34.3 |
| | | EPO(67%) | solvent | microns | | 16.4 | 19.4 | 20.2 |
| | | Sodium | | | | | | |
| | | bicarb(33%) | | | | | | |
| 43 | 48%/48% | Eudragit EPO | Disk- | 20-110 | | 17.3 | 28.0 | 51.0 |
| | | (61.5%) PEG | solvent | microns | | 22.2 | 24.9 | 50.3 |
| | | 300(11.5%) | | | | 27.8(pH5) | 1.9(pH5) | 0(pH5) |
| | | PEG 3350 | | | | 22.4(pH5) | 1.7(pH5) | 0(pH5) |
| | | (3.8%) Sod | | | | 59.2(pH6) | 41.4(pH6) | 25.2(pH6) |
| | | bicarb(23.2%) | | | | 55.8(pH6) | 39.5(pH6) | 23.9(pH6) |
| 44 | 70%/66% | Eudragit EPO | Disk- | 20-100 | | 21.7 | 27.1 | 43.6 |
| | | | solvent | microns | | 21.3 | 25.2 | 54.4 |
| | | | | | | 27.8(pH5) | 0.9(pH5) | 0(pH5) |
| | | | | | | 17.8(pH5) | 0.5(pH5) | 0(pH5) |
| | | | | | | 59.1(pH6) | 39.0(pH6) | 23.6(pH6) |
| | | | | | | 31.6(pH6) | 38.4(pH6) | 22.8(pH6) |
| 45 | 25%/26% | Opadry AMB | Spray dry | | | 90.0 | 84.1 | 79.8 |
| | | (No TiO₂) | | | | 87.1 | 84.6 | 79.6 |
| 46 | 25%/24% | Opadry AMB | Spray dry | | | 56.2 | 85.0 | 81.6 |
| | | (No TiO₂) | | | | 90.0 | 85.8 | 81.7 |
| 47 | 25%/24% | Opadry AMB | Spray dry | | | 93.4 | 90.0 | 86.8 |
| | | (No TiO₂) | | | | 88.9 | 87.5 | 82.7 |
| | | BHT (0.1%) | | | | | | |
| 51 | 66%/ | Eudragit EPO | Disk- | 20-100 | | 21.7 | 27.1 | 43.6 |
| | | | solvent | microns | | 21.3 | 25.2 | 54.4 |
| 52 | 24%/ | Opadry AMB | Spray Dry | 5-30 | | 93.4 | 90.0 | 86.8 |
| | | BHT | (aqueous) | microns | | 88.9 | 87.5 | 82.7 |

### Example 2

### Preparation of Chewable Tablets

The chart below summarizes the wt %, the feed rates used, and the inlet/outlet temperatures for eleven different omeprazole microspheres.

The tablets were manufactured using the following materials: Encapsulated omeprazole (varied based on payload, to deliver 40 mg potency), sodium bicarbonate (1260 mg), calcium carbonate (790 mg), croscarmellose sodium (64 mg), Klucel (160 mg), Xylitab 100 (380 mg), microcrystalline cellulose (128 mg), sucralose (162 mg), peppermint durarome (34 mg), peach flavor (100 mg), masking powder (60 mg), FD&C Lake No. 40 Red (3 mg), and magnesium stearate (32 mg).

The amount of encapsulated omeprazole used in each tablet batch varies based on the actual payload of each set of microcapsules to achieve the theoretical dose of 40 mg. The omeprazole was microencapsulated in a similar manner as that described in Example 1. All ingredients are mixed well to achieve a homogenious blend.

Tablets containing omeprazole microspheres were prepared using a high-speed rotary tablet press (TBCB Pharmaceutical Equipment Group, Model ZPY15). Round, convex tablets with diameters of about 10 mm and an average weight of approximately 600 mg per tablet were prepared.

An exemplary formulation used to make each of the tablets, as well as the blending methods used, are shown below:

| | Method and | Microencapsulation | Wt % of | Feed Rate | Inlet/Outlet |
|---|---|---|---|---|---|
| Sample | Solvent | Material | material | (g/min) | Temp(° C.) |
| 53 | Spray dry* | Methocel A15 LV | 5% | 4.2 | 125/70 |
| | Water | PEG 3350 | | | |
| 54 | Spray dry | Methocel A15 LV | 5% | 4.0 | 125/70 |
| | Water | BHT | | | |
| 55 | Spray dry | Opadry YS-1-7003 | 5% | 4.2 | 126/60 |
| | Water | PEG 3350 | | | |
| | | BHT | | | |
| 56 | Spray dry | Kollicoat IR | 10% | 3.0 | 128/85 |
| | Water | PEG 3350 | | | |
| | | BHT | | | |
| 57 | Spray dry | Eudragit RD100 | 5% | 4.0 | 127/87 |
| | Water | PEG 3350 | | | |
| | | BHT | | | |
| 58 | Spray dry | Klucel | 5% | 4.2 | 126/83 |
| | Water | PEG 3350 | | | |
| | | BHT | | | |
| 59 | Spinning disk** | Klucel | 10% | 90 | /52 |
| | 75% Methanol | | | | |
| | 25% Acetone | | | | |
| 60 | Spray dry | Kollicoat | 5% | 4.5 | 129/86 |
| | Water | Sodium Bicarb | | | |
| 61 | Spray dry | Klucel | 5% | 4.5 | 122/84 |
| | Water | Sodium Bicarb | | | |
| 62 | Spinning disk | Eudragit EPO | 10% | 90 | /50 |
| | 75% Methanol | | | | |
| | 25% Acetone | | | | |
| 63 | Spray dry | Opadry AMB | 10% | 4.4 | 124/79 |
| | Water | BHT | | | |

*Used a concentric nozzle with 0.055 inch air opening and a 0.028 inch fluid opening.
**Used a 3-inch stainless steel disk rotating at approximately 4,500 rpm.

### Example 3

### Preparation of Chewable Tablets

Various tablets were manufactured using the following materials: Encapsulated omeprazole (varied based on payload, to deliver 40 mg potency), sodium bicarbonate (600 mg), MS-95 (5% starch) (737 mg), croscarmellose sodium (33 mg), Klucel (90 mg), Xylitab 100 (200 mg), sucralose (80 mg), peppermint durarome (10 mg), peach flavor (52 mg), masking powder (27 mg), Lake FD & C Red #40 (2 mg), and magnesium stearate (17 mg).

### Example 4

### Preparation of Capsule Containing Omeprazole Microgranules

The capsule product is manufactured using the following materials: Encapsulated omeprazole (varied based on payload, to deliver 40 mg potency), sodium bicarbonate (200 mg), magnesium hydroxide (600 mg), croscarmellose sodium (50 mg), Klucel (50 mg), and magnesium stearate (5 mg).

The amount of encapsulated omeprazole used in each tablet batch varies based on the actual payload of each set of microcapsules to achieve the theoretical dose of 40 mg. The omeprazole was microencapsulated in a similar manner as that described in Example 1. All ingredients are mixed well to achieve a homogenous bulk blend which is then filled into a hard gelatine capsule such as a size 00 gelatine capsule from Capsugel.

### Example 5

### Tablets Used in Stability Studies

Various tablets used in the stability studies were manufactured using the following materials: Encapsulated omeprazole (varied based on payload, see below), sodium bicarbonate (1260 mg), calcium carbonate (790 mg), croscarmellose sodium (64 mg), Klucel (160 mg), Xylitab 100 (380 mg), microcrystalline cellulose (128 mg), sucralose (162 mg), peppermint duraromer)34 mg), peach duraromer (100 mg), masking powder (60 mg), FD&C Lake No. 40 Red (3 mg), and magnesium stearate (32 mg).

The table below shows the payload of various microencapsules, the amount of omeprazole, and shell material used.

| | Omeprazole | | |
|---|---|---|---|
| | payload in | | |
| | microcapsule | | |
| | (Theoretical/ | Microsphere | Mg of Omeprazole |
| Sample | Analytical) | shell material | per gram of tablet |
| 64 | 25%/26.2% | Kollicoat IR | 12.03 |
| | | PEG 3350 (10%) | |
| | | BHT (0.1%) | |
| 65 | 25%/23.3% | Methocel A15 LV | 11.96 |
| | | PEG 3350 (5%) | |
| 66 | 25%/20.5% | Opadry YS-1-7003 | 11.88 |
| | | PEG 3350 (5%) | |
| | | BHT (0.1%) | |
| 67 | 25%/24.8% | Methocel A15 LV | 12.00 |
| | | BHT (0.1%) | |
| 68 | 25%/26.0% | Kollicoat IR | 12.02 |
| | | Sodium bicarbonate | |
| 69 | 25%/26.3% | Klucel | 12.03 |
| | | Sodium bicarbonate | |
| 70 | 25%/21.3% | Eudragit RD100 | 11.90 |
| | | PEG 3350 (5%) | |
| | | BHT (0.1%) | |
| 71 | 25%/26.0% | Klucel | 12.02 |
| | | PEG 3350 (5%) | |
| | | BHT (0.1%) | |
| 72 | 25%/24.7% | Opadry AMB | 11.99 |
| 73 | 70%/66.1% | Eudragit EPO | 12.37 |
| Placebo | Not Applicable | Not Applicable | 10.00 |

### Example 6

### Analytical Assay for Determining the Amount of Omeprazole Present in Tablets Containing Omeprazole Microspheres

The following procedure was used to determine the potency of omeprazole in the tablets. The tablet was accurately weighed and placed into 100 ml volumetric flask. To that, 1.0 ml of Nanopure water was added to wet and soften the tablet. The solution was allowed to stand for 30 minutes. After sitting, the sample was vortexed and sonicated for 30 minutes or until completely dissolved. 1.0 ml of chloroform was then added and the sample was vortexed and sonicated for an additional 15 minutes. The solution was then brought to volume with methanol and vortexed again to mix solution. 10 ml was then decanted into a 10 cc syringe fitted with a 0.45-micron filter. The material was pushed through the filter and the first several milliliters were discarded. The remaining mixture was then collected for HPLC injection. A 5-point calibration curve was prepared in methanol ranging from 15 to 300 μg/ml. The following chromatographic conditions were used mobile phase: 75.5% Na₂PO₄, pH=8.0, 24.5% acetonitrile; flow rate: 1.0 mL/min; injection volume: 20 μL; detector: UV, 280 nm; column: waters symmetry shield RP8.

### Example 7

### Stability Study of Microencapsulated Omeprazole

Microspheres that exhibited dissolution results with greater than 80% omeprazole release after 2 hours were placed on stability. The microspheres were stored in opened vials at 25° C. All samples showed degradation after 4 weeks at elevated temperatures. The open vials stored at 25° C. were analyzed after 6-8 weeks for potency and for impurities using the Omeprazole EP method. The stability results are summarized in the table below.

| | Omeprazole | 4-Week Potency Values | AUC |
|---|---|---|---|
| Sample | Loading (Initial) | (Omeprazole Loading) | Purity* |
| 5 | 23.3 | 25.0(107% of initial)@25° C. | 95.65 |
| 6 | 26.0 | 24.9(95.8% of initial) @25° C. | 99.90 |
| 8 | 24.8 | 26.4(106.6% of initial)@25° C. | 99.95 |
| 10 | 2.2 | 2.3(106% of initial) @25° C. | 76.16 |
| 11 | 20.5 | 22.6(110% of initial) @25° C. | 100.0 |
| 13 | 26.2 | 23.8(90.8% of initial) @25° C. | 99.54 |
| 14 | 21.3 | 19.1(89.5% of initial) @25° C. | 98.88 |
| 15 | 26.0 | 22.8(87.8% of initial)@25° C. | 99.70 |
| 17 | 25.8 | 21.9(84.9% of initial) @25° C. | 98.22 |
| | | | (99.3@T₀) |
| 23 | 22.2 | 20.7 (93.2% of initial) @25° C. | 97.69 |
| 35 | 26.0 | 21.7(83.6% of initial) @25° C. | 97.88 |

*AUC Purity = Area Under the Curve after 6-8 weeks at 25° C. in open container.

### Example 8

### Method for Determining Payload of Omeprazole Microspheres

The HPLC samples for the omeprazole assay of various microspheres were prepared as follows: 5 mg of the microsphere were accurately weighed into a screw cap culture tube. To that, 200 μL of chloroform was added. The microspheres were allowed to dissolve, sonicated and vortex for approximately one minute. Then, 10 ml of methanol was added and the sample was again vortexed for one minute. Once completed, an aliquot of the sample was removed for HPLC analysis.

A 5-point calibration curve was prepared in methanol ranging from 20 to 500 μg/mL to calculate payload. The chromatographic conditions were: Mobile phase: 75.5% Na₂PO₄ pH 8.0, 24.5% Acetonitrile; Flow Rate: 1.0 mL/min; Run Time: 15 min; Injection Volume: 20 μL; Detector: U.V., 280 nm; Column: Waters SymmetryShield RP8.

### Example 9

### Method for Determining the Amount of Impurities Present in the Microspheres

The HPLC samples for the omeprazole assay of various microspheres were prepared in the following manner. 5 mgs of the omeprazole microspheres were weighed into a screw cap culture tube. To that, 200 μL of chloroform were added. The microspheres were allowed to dissolve, sonicate and vortex for approximately one minute each. 10 mL of methanol was then added and the sample was again vortexed for 1 minute. Once complete, an aliquot was removed for HPLC analysis.

For standards, 100 μg/mL concentration of omeprazole in methanol for a marker was prepared. A 0.1 μg/mL concentration of omeprazole was then prepared to set one-half the minimal detection limit. Then, a 1 μg/mL concentration of omeprazole impurity D in methanol was prepared. The chromatographic conditions were: Mobile Phase: 75% Na₂PO₄ pH 7.6, 25% acetonitrile; Flow Rate: 1.0 mL/min; Run Time: 30 min; Injection Volume: 20 μL; Detector: U.V., 280 nm; Column: Waters SymmetryShield RP8.

### Example 10

### Method for Determining Dissolution of Omeprazole Microspheres

The omeprazole potency method was used for the dissolution testing. The HPLC samples for the omeprazole assay of various microspheres were prepared according to the following method. 5 mgs of the microspheres were accurately weighed into an 8 ounce amber bottle. To that, 100 ml of pH 7.4 monobasic phosphate buffer was added. The samples were placed in a 37° C. water bath and vigorously shaken until the end of the release study. Using an Eppendorf pipette, 100 μL was removed and the outside part of the tip was rinsed with 100 μL of buffer back into the sample bottle. The sample was then transferred into a limited insert for HPLC analysis using a 1 cc syringe fitted with a 45 micron filter. Samples were then taken at 30, 45, and 120 minutes.

A 6-point calibration curve was prepared in diluent (70% sodium phosphate pH 10.0/30% acetonitrile) ranging from 1 to 120 μg/mL to determine sample release rates. The chromatographic conditions were: Mobile phase: 75.5% Na₂PO₄ pH 8.0, 24.5% Acetonitrile; Flow Rate: 1.0 mL/min; Run Time: 15 min; Injection Volume: 20 μL; Detector: U.V., 280 nm; Column: Waters SymmetryShield RP8.

### Example 11

### Optical Microscopy

The omeprazole microspheres were observed using an Olympus BX60 optical microscope equipped with an Olympus DP10 digital camera to determine their particle size and morphology characteristics. The microspheres were observed at either 100× or 200× magnification.

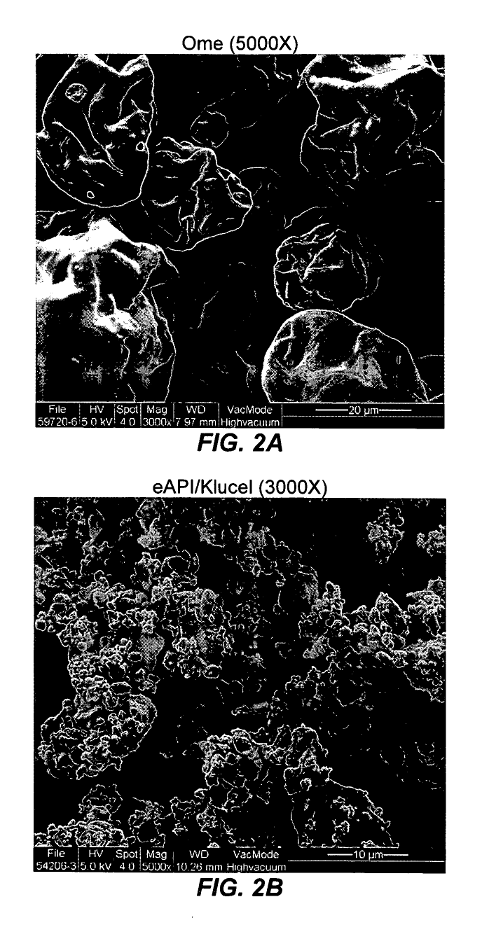

The microspheres prepared by spray drying were in the size range of 5 to 30 microns. The microspheres prepared by spinning disk-solvent process were in the size range of 25 to 100 microns. The microspheres prepared by spinning disk-hot melt process were in the size range of 30 to 125 microns. See FIG. 2.

### Example 12

### Thermal Gravimetric Analysis (TGA)

Thermal Gravimetric Analysis was performed on neat omeprazole (Two lots from Uquifa and USP Standard) and the omeprazole microspheres using a TA Instruments Model 2950 equipped with Thermal Solutions Instrument Software and Universal Analysis Data Software. The neat omeprazole samples showed very little weight loss up to 150° C. at which temperature a dramatic weight loss begins. This weight loss occurs at the melting point of omeprazole which is in the range of 150-160° C.

For the omeprazole microspheres, the percent weight loss up to 140° C. was recorded to determine the amount of volatiles present. Most samples exhibit a weight loss of less than 1% up to 140° C. except the samples that contained sodium bicarbonate which have a greater weight loss, from 7-32%. The following TGA run conditions were used: nitrogen atmosphere; Isothermal for 5 minutes at 25° C.; ramp 10° C./minute to 250° C.; platinum sample pan.

Many modifications, equivalents, and variations of the present invention are possible in light of the above teachings, therefore, it is to be understood that within the scope of the appended claims, the invention may be practiced other than as specifically described.

1. A pharmaceutical formulation having an enhanced shelf-life, comprising:
(a) at least one acid labile proton pump inhibitor which is microencapsulated with a material that enhances the shelf-life of the pharmaceutical formulation; and
(b) at least one antacid;
wherein an initial serum concentration of the proton pump inhibitor is greater than about 0.1 μg/ml at any time within about 30 minutes after administration of the pharmaceutical formulation.

2. A pharmaceutical formulation according to claim 1, wherein the proton pump inhibitor is a substituted bicyclic aryl-imidazole selected from the group consisting of omeprazole, hydroxyomeprazole, esomeprazole, tenatoprazole, lansoprazole, pantoprazole, rabeprazole, dontoprazole, habeprazole, perprazole, ransoprazole, pariprazole, leminoprazole; or a free base, free acid, salt, hydrate, ester, amide, enantiomer, isomer, tautomer, polymorph, or prodrug thereof.

3. A pharmaceutical formulation according to claim 1, wherein the proton pump inhibitor is selected from omeprazole, lansoprazole, esomeprazole, or a free base, free acid, salt, hydrate, ester, amide, enantiomer, isomer, tautomer, polymorph, or prodrug thereof.

4. A pharmaceutical formulation according to claim 1 comprising about 5 mgs to about 200 mgs of the proton pump inhibitor.

5. A pharmaceutical formulation according to claim 1 comprising about 10 mgs, or about 15 mgs, or about 20 mgs, or about 30 mgs, or about 40 mgs, or about 60 mgs of the proton pump inhibitor.

6. A pharmaceutical formulation according to claim 1, wherein the antacid is an alkaline metal salt or a Group IA metal selected from a bicarbonate salt of a Group IA metal, a carbonate salt of a Group IA metal.

7. A pharmaceutical formulation according to claim 1, wherein the antacid is selected from sodium bicarbonate, sodium carbonate, calcium carbonate, magnesium oxide, magnesium hydroxide, magnesium carbonate, aluminum hydroxide, and mixtures thereof.

8. A pharmaceutical formulation according to claim 1, wherein the antacid comprises at least one soluble buffer.

9. A pharmaceutical formulation according to claim 8, wherein the soluble buffer is present in at least about 5 mEq.

10. A pharmaceutical formulation according to claim 1 comprising about 500 to about 2000 mg of antacid.

11. A pharmaceutical formulation according to claim 1, wherein the material that enhances the shelf-life of the pharmaceutical formulation is selected from the group consisting of cellulose hydroxypropyl ethers; low-substituted hydroxypropyl ethers; cellulose hydroxypropyl methyl ethers; methylcellulose polymers; ethylcelluloses and mixtures thereof; polyvinyl alcohol; hydroxyethylcelluloses; carboxymethylcelluloses and salts of carboxymethylcelluloses; polyvinyl alcohol and polyethylene glycol co-polymers; monoglycerides; triglycerides; polyethylene glycols, modified food starch, acrylic polymers; mixtures of acrylic polymers with cellulose ethers; cellulose acetate phthalate; sepifilms, cyclodextrins; and mixtures thereof.

12. A pharmaceutical formulation according to claim 1, wherein the material that enhances the shelf-life of the pharmaceutical composition is a cellulose hydroxypropyl ether.

13. A pharmaceutical formulation according to claim 1, wherein the material that enhances the shelf-life of the pharmaceutical composition is a mixture of methylcellulose and hydroxypropyl and methylcellulose polymers.

14. A pharmaceutical formulation according to claim 1, wherein the microencapsulated proton pump inhibitor has less than 1% degradation after one month of storage at room temperature.

15. A pharmaceutical formulation according to claim 1, wherein the pharmaceutical formulation has less than 5% total impurities after 1 year of storage at room temperature.

16. A pharmaceutical formulation according to claim 1, wherein after 3 years of storage at room temperature, the pharmaceutical formulation of claim 1 has less degradation than an equivalent pharmaceutical formulation comprising non-microencapsulated proton pump inhibitor.

17. A pharmaceutical formulation according to claim 1 further comprising one or more excipients selected from the group consisting of parietal cell activators, organic solvents, erosion facilitators, flavoring agents, sweetening agents, diffusion facilitators, antioxidants and carrier materials selected from binders, suspending agents, disintegration agents, filling agents, surfactants, solubilizers, stabilizers, lubricants, wetting agents, diluents, anti-adherents, and antifoaming agents.

18. A pharmaceutical formulation according to claim 17, wherein the flavoring agent is selected from is selected from peach, menthol, aspartame, sucralose, sucrose, and monoammonium gylcyrhizinate.

19. A pharmaceutical formulation according to claim 17, wherein the suspending agent is selected from xantham gum, povidone, guar gum, and hydroxypropyl methylcellulose.

20. A pharmaceutical formulation according to claim 1 the form of a capsule, a chewable tablet, a tablet, or a powder.

21. A pharmaceutical formulation according to claim 1, wherein an initial serum concentration of the proton pump inhibitor is greater than about 0.5 μg/ml at any time within about 1 hour after administration of the pharmaceutical formulation.

22. A pharmaceutical formulation according to claim 1, wherein the maximum serum concentration is reached within about 1 hour after administration of the pharmaceutical formulation.

23. A pharmaceutical formulation according the claim 1, wherein the average particle size of the microencapsulated proton pump inhibitor is between about 20 to about 500 microns in diameter.

24. A pharmaceutical formulation according the claim 1, wherein the average particle size of the microencapsulated proton pump inhibitor is between about 50 to about 150 microns in diameter.

25. A pharmaceutical formulation according to claim 1, wherein the average particle size of the microencapsulated proton pump inhibitor is less than about 150 microns in diameter.

26. A taste-masked pharmaceutical formulation comprising:
(a) at least one acid labile proton pump inhibitor which is microencapsulated with a taste-masking material; and
(b) at least one antacid;
wherein an initial serum concentration of the proton pump inhibitor is greater than about 0.1 μg/ml at any time within about 30 minutes after administration of the pharmaceutical formulation.

27. A pharmaceutical formulation according to claim 26, wherein the proton pump inhibitor is a substituted bicyclic aryl-imidazole selected from the group consisting of omeprazole, hydroxyomeprazole, esomeprazole, tenatoprazole, lansoprazole, pantoprazole, rabeprazole, dontoprazole, habeprazole, perprazole, ransoprazole, pariprazole, leminoprazole; or a free base, free acid, salt, hydrate, ester, amide, enantiomer, isomer, tautomer, polymorph, or prodrug thereof.

28. A pharmaceutical formulation according to claim 26, wherein the proton pump inhibitor is selected from omeprazole, lansoprazole, esomeprazole, or a free base, free acid, salt, hydrate, ester, amide, enantiomer, isomer, tautomer, polymorph, or prodrug thereof.

29. A pharmaceutical formulation according to claim 1 comprising about 5 mgs to about 200 mgs of the proton pump inhibitor.

30. A pharmaceutical formulation according to claim 26 comprising about 10 mgs, or about 15 mgs, or about 20 mgs, or about 30 mgs, or about 40 mgs, or about 60 mgs of the proton pump inhibitor.

31. A pharmaceutical formulation according to claim 26, wherein the antacid is an alkaline metal salt or a Group IA metal selected from a bicarbonate salt of a Group IA metal, a carbonate salt of a Group IA metal.

32. A pharmaceutical formulation according to claim 26, wherein the antacid is selected from sodium bicarbonate, sodium carbonate, calcium carbonate, magnesium oxide, magnesium hydroxide, magnesium carbonate, aluminum hydroxide, and mixtures thereof.

33. A pharmaceutical formulation according to claim 26, wherein the antacid comprises at least one soluble buffer.

34. A pharmaceutical formulation according to claim 33, wherein the soluble buffer is present in at least about 5 mEq.

35. A pharmaceutical formulation according to claim 26 comprising about 500 to about 2000 mg of antacid.

36. A pharmaceutical formulation according to claim 26, wherein the taste-masking material is selected from the group consisting of cellulose hydroxypropyl ethers; low-substituted hydroxypropyl ethers; cellulose hydroxypropyl methyl ethers; methylcellulose polymers; ethylcelluloses and mixtures thereof; polyvinyl alcohol; hydroxyethylcelluloses; carboxymethylcelluloses and salts of carboxymethylcelluloses; polyvinyl alcohol and polyethylene glycol co-polymers; monoglycerides; triglycerides; polyethylene glycols, modified food starch, acrylic polymers; mixtures of acrylic polymers with cellulose ethers; cellulose acetate phthalate; sepifilms, cyclodextrins; and mixtures thereof.

37. A pharmaceutical formulation according to claim 26, wherein the taste-masking material is a cellulose hydroxypropyl ether.

38. A pharmaceutical formulation according to claim 26, wherein the taste-masking material is a mixture of methylcellulose and hydroxypropyl and methylcellulose polymers.

39. A pharmaceutical formulation according to claim 26 further comprising one or more excipients selected from the group consisting of parietal cell activators, organic solvents, erosion facilitators, flavoring agents, sweetening agents, diffusion facilitators, antioxidants and carrier materials selected from binders, suspending agents, disintegration agents, filling agents, surfactants, solubilizers, stabilizers, lubricants, wetting agents, diluents, anti-adherents, and antifoaming agents.

40. A pharmaceutical formulation according to claim 39, wherein the flavoring agent is selected from is selected from peach, menthol, aspartame, sucralose, sucrose, and monoammonium gylcyrhizinate.

41. A pharmaceutical formulation according to claim 39, wherein the suspending agent is selected from xantham gum, povidone, guar gum, and hydroxypropyl methylcellulose.

42. A pharmaceutical formulation according to claim 26 in the form of a capsule, a chewable tablet, a tablet, or a powder.

43. A pharmaceutical formulation according to claim 26, wherein an initial serum concentration of the proton pump inhibitor is greater than about 0.5 μg/ml at any time within about 1 hour after administration of the pharmaceutical formulation.

44. A pharmaceutical formulation according to claim 26, wherein the maximum serum concentration is reached within about 1 hour after administration of the pharmaceutical formulation.

45. A pharmaceutical formulation according the claim 26, wherein the average particle size of the microencapsulated proton pump inhibitor is between about 20 to about 500 microns in diameter.

46. A pharmaceutical formulation according the claim 26, wherein the average particle size of the microencapsulated proton pump inhibitor is between about 50 to about 150 microns in diameter.

47. A pharmaceutical formulation according to claim 26, wherein the average particle size of the microencapsulated proton pump inhibitor is less than about 150 microns in diameter.

48. A pharmaceutical formulation according to claim 26, wherein the taste-masking material is less than about 50% of the total weight composition.

49. A pharmaceutical formulation according to claim 26, wherein the amount of flavoring agent necessary to create a palatable product is decreased by at least about 20%, as compared to a pharmaceutical formulation comprising non-microencapsulated proton pump inhibitor.

50. A pharmaceutical formulation according to claim 26, wherein the amount of flavoring agent necessary to create a palatable product is decreased, as compared to a pharmaceutical formulation comprising non-microencapsulated proton pump inhibitor.

51. A pharmaceutical formulation according to claim 26 comprising less than about 2 grams of flavoring agent.

52. A method of extending the shelf-life of a pharmaceutical formulation comprising:
(a) microencapsulating at least one acid labile proton pump inhibitor with a material that enhances the shelf-life; and
(b) combining the microencapsulated acid labile proton pump inhibitor with at least one antacid.

53. A method of masking the taste of a pharmaceutical formulation comprising:
(a) microencapsulating at least one acid labile proton pump inhibitor with a taste-masking material; and
(b) combining the microencapsulated acid labile proton pump inhibitor with an antacid.

54. A method of treating an acid related gastrointestinal disorder in a subject in need thereof by administering the pharmaceutical formulation of claim 1.

55. A method of treating an acid related gastrointestinal disorder in a subject in need thereof by administering the pharmaceutical formulation of claim 26.
